(19) United States
(12) Patent Application Publication
(10) Pub. No.: US 2023/0312718 A1
(43) Pub. Date: Oct. 5, 2023

(54) Methods of Treating Recurrent Ovarian Cancer with Bispecific Anti-MUC16 x Anti-CD3 Antibodies Alone or in Combination with Anti-PD-1 Antibodies

(71) Applicant: Regeneron Pharmaceuticals, Inc, Tarrytown, NY (US)

(72) Inventors: Glenn Kroog, New York, NY (US); Tracey Michelle Rowlands, Mamaroneck, NY (US); Priscila Hermont Barcellos Goncalves, Greenvale, NY (US); Thomas Smith Uldrick, JR., Pelham, NY (US); Min Zhu, Ossining, NY (US)

(21) Appl. No.: 18/094,176

(22) Filed: Jan. 6, 2023

Related U.S. Application Data
(60) Provisional application No. 63/421,541, filed on Nov. 1, 2022.
(60) Provisional application No. 63/394,483, filed on Aug. 2, 2022.
(60) Provisional application No. 63/342,542, filed on May 16, 2022.
(60) Provisional application No. 63/297,333, filed on Jan. 7, 2022.

(51) Int. Cl. C07K 16/28

(52) U.S. Cl. CPC C07K 16/2809; C07K 16/2878; A61K 2039/507

(57) ABSTRACT

The present invention provides methods for treating, reducing the severity, or inhibiting the growth of cancer (e.g., recurrent ovarian cancer). The methods of the present invention comprise administering to a subject in need thereof a therapeutically effective amount of a bispecific antibody that specifically binds Mucin 16 (MUC16) and CD3 alone, or in combination with a therapeutically effective amount of an antibody or antigen-binding fragment thereof that specifically binds to programmed death 1 (PD-1) receptor.

### CROSS-REFERENCE TO RELATED APPLICATIONS

This application claims the benefit under 35 USC §119(e) of U.S. Provisional Application Nos.: 63/297,333, filed Jan. 7, 2022; 63/342,542, filed May 16, 2022; 63/394,483, filed Aug. 2, 2022; and 63/421,541, filed Nov. 1, 2022, each of which is incorporated herein by reference in its entirety for all purposes.

### REFERENCE TO A SEQUENCE LISTING

This application incorporates by reference a computer readable Sequence Listing in ST.26 XML format, titled 11048US01_Sequence, created on Jan. 6, 2023 and containing 54,660 bytes.

### FIELD OF THE INVENTION

The present invention relates to methods for treating cancer with a bispecific antibody that binds to mucin 16 (MUC16) and CD3, alone or in combination with an anti-PD-1 antibody.

### BACKGROUND

Mucin 16 (MUC16), also known as cancer antigen 125, carcinoma antigen 125, carbohydrate antigen 125, or CA-125, is a single transmembrane domain highly glycosylated integral membrane glycoprotein that is highly expressed in ovarian cancer. MUC16 consists of three major domains: an extracellular N-terminal domain, a large tandem repeat domain interspersed with sea urchin sperm, enterokinase, and agrin (SEA) domains, and a carboxyl terminal domain that comprises a segment of the transmembrane region and a short cytoplasmic tail. Proteolytic cleavage results in shedding of the extracellular portion of MUC16 into the bloodstream. MUC16 is overexpressed in cancers including ovarian cancer, breast cancer, pancreatic cancer, non-small-cell lung cancer, intrahepatic cholangiocarcinoma-mass forming type, adenocarcinoma of the uterine cervix, and adenocarcinoma of the gastric tract, and in diseases and conditions including inflammatory bowel disease, liver cirrhosis, cardiac failure, peritoneal infection, and abdominal surgery. (Haridas, D. et al., 2014, FASEB J., 28:4183-4199). Expression on cancer cells is shown to protect tumor cells from the immune system. (Felder, M. et al., 2014, Molecular Cancer, 13:129) Methods for treating ovarian cancer using antibodies to MUC16 have been investigated. Oregovomab and abgovomab are anti-MUC16 antibodies which have had limited success. (Felder, supra, Das, S. and Batra, S.K. 2015, Cancer Res. 75:4660-4674.)

CD3 is a homodimeric or heterodimeric antigen expressed on T cells in association with the T cell receptor complex (TCR) and is required for T cell activation. Functional CD3 is formed from the dimeric association of two of four different chains: epsilon, zeta, delta and gamma. The CD3 dimeric arrangements include gamma/epsilon, delta/epsilon and zeta/zeta. Antibodies against CD3 have been shown to cluster CD3 on T cells, thereby causing T cell activation in a manner similar to the engagement of the TCR by peptide-loaded MHC molecules. Thus, anti-CD3 antibodies have been proposed for therapeutic purposes involving the activation of T cells. In addition, bispecific antibodies that are capable of binding CD3 and a target antigen have been proposed for therapeutic uses involving targeting T cell immune responses to tissues and cells expressing the target antigen.

Programmed death receptor-1 (PD-1) signaling in the tumor microenvironment plays a key role in allowing tumor cells to escape immune surveillance by the host immune system. Blockade of the PD-1 signaling pathway has demonstrated clinical activity in patients with multiple tumor types, and antibody therapeutics that block PD-1 (e.g., nivolumab and pembrolizumab) have been approved for the treatment of metastatic melanoma and metastatic squamous non-small cell lung cancer. Recent data has demonstrated the clinical activity of PD-1 blockade in patients with aggressive NHL and Hodgkin’s lymphoma (Lesokhin, et al. 2014, Abstract 291, 56th ASH Annual Meeting and Exposition, San Francisco, Calif.; Ansell et al. 2015, N. Engl. J. Med. 372(4):311-9).

Ovarian cancer is the most lethal of the gynecologic malignancies; although the estimated number of new cases of ovarian cancer among American women are much lower than certain other cancers, the death-to-incidence ratio for ovarian cancer is considerably higher (Siegal et al., CA Cancer J Clin 66:7-30, 2016). Ovarian cancer is frequently diagnosed at an advanced stage, which contributes to its lethality. The current standard of care for ovarian cancer is surgery followed by chemotherapy, namely a combination of platinum agents and taxanes. Whilst the majority of patients respond to initial treatment, most experience a recurrence of the disease, resulting in a cycle of repeated surgeries and additional rounds of chemotherapy. Although recurrent ovarian cancers may respond to further treatment, virtually all of them will ultimately become resistant to currently available therapies. Despite recent advances in therapy such as PARP inhibitors for patients carrying BRCA or other homologous recombination deficiency (HRD) mutations, advanced ovarian cancer remains a disease of high unmet need.

Evidence suggests that ovarian cancer may be amenable to some forms of immunotherapy (Kandalaft et al., J. Clin. Oncol., 29:925-933, 2011). For example, ovarian cancer patients whose tumors were positive for intraepithelial CD8⁺ T lymphocyte infiltration had significantly better overall and progression-free survival than patients without intraepithelial CD8⁺ T lymphocyte infiltration (Hamanishi et al., PNAS, 104:3360-65, 2007; and Zhang et al., N. Engl. J. Med., 348:203-213, 2003). Moreover, some patients have shown spontaneous immune response to their tumors, demonstrated by detection of tumor-reactive T cells and antibodies in the blood, tumor or ascites of patients with advanced disease (Schliengar et al., Clin Cancer Res, 9:1517-1527, 2003). Blockade of the PD-⅟ PD-L1/ checkpoint pathway has shown some benefit in ovarian cancer; PD-1 blockade monotherapy resulted in an overall response rate (ORR) of approximately 10-15% in early clinical trials (Hamanishi et al., supra). However, blockade of this pathway alone is clearly not sufficient.

In view of the high unmet need, additional therapies for targeting ovarian cancer are needed.

### BRIEF SUMMARY OF THE INVENTION

In one aspect, the present disclosure includes a method of treating a cancer (e.g., a MUC16-expressing cancer) in a subject in need thereof, comprising administering to the subject a bispecific antibody or antigen-binding fragment thereof comprising a first antigen-binding domain that specifically binds mucin 16 (MUC16) on a target tumor cell, and a second antigen-binding domain that specifically binds human CD3 on a T cell. In some embodiments, the bispecific antibody or antigen-binding fragment thereof is administered to the subject at a dose of at least 1 mg (e.g., weekly).

In some embodiments, the cancer (e.g., MUC16-expressing cancer) is ovarian cancer, fallopian tube cancer, or primary peritoneal cancer. In some cases, the cancer (e.g., MUC16-expressing cancer) is resistant to platinum-based chemotherapy. In some cases, the subject has previously been treated with a platinum-based chemotherapy.

In some embodiments, the bispecific antibody or antigen-binding fragment thereof comprises a first antigen-binding domain comprising: (a) three heavy chain complementarity determining regions (HCDR1, HCDR2 and HCDR3) contained within a heavy chain variable region (HCVR) comprising the amino acid sequence of SEQ ID NO: 1; and (b) three light chain complementarity determining regions (LCDR1, LCDR2 and LCDR3) contained within a light chain variable region (LCVR) comprising the amino acid sequence of SEQ ID NO: 2. In some cases, the first antigen-binding domain comprises a HCDR1 comprising the amino acid sequence of SEQ ID NO: 8, a HCDR2 comprising the amino acid sequence of SEQ ID NO: 9, and a HCDR3 comprising the amino acid sequence of SEQ ID NO: 10. In some cases, the first antigen-binding domain comprises a LCDR1 comprising the amino acid sequence of SEQ ID NO: 11, a LCDR2 comprising the amino acid sequence of SEQ ID NO: 12, and a LCDR3 comprising the amino acid sequence of SEQ ID NO: 13. In some cases, the first antigen-binding domain comprises a HCVR comprising the amino acid sequence of SEQ ID NO: 1, and a LCVR comprising the amino acid sequence of SEQ ID NO: 2.

In some embodiments, including those in which the first antigen-binding domain is as discussed above, the bispecific antibody or antigen-binding fragment thereof comprises a second antigen-binding domain comprising: (a) three heavy chain complementarity determining regions (HCDR1, HCDR2 and HCDR3) contained within a heavy chain variable region (HCVR) comprising the amino acid sequence of SEQ ID NO: 3; and (b) three light chain complementarity determining regions (LCDR1, LCDR2 and LCDR3) contained within a light chain variable region (LCVR) comprising the amino acid sequence of SEQ ID NO: 2. In some cases, the second antigen-binding domain comprises a HCDR1 comprising the amino acid sequence of SEQ ID NO: 14, a HCDR2 comprising the amino acid sequence of SEQ ID NO: 15, and a HCDR3 comprising the amino acid sequence of SEQ ID NO: 16. In some cases, the second antigen-binding domain comprises a LCDR1 comprising the amino acid sequence of SEQ ID NO: 11, a LCDR2 comprising the amino acid sequence of SEQ ID NO: 12, and a LCDR3 comprising the amino acid sequence of SEQ ID NO: 13. In some cases, the second antigen-binding domain comprises a HCVR comprising the amino acid sequence of SEQ ID NO: 3, and a LCVR comprising the amino acid sequence of SEQ ID NO: 2.

In some embodiments, the bispecific antibody comprises a human IgG heavy chain constant region. In some cases, the human IgG heavy chain constant region is isotype IgG1. In some cases, the human IgG heavy chain constant region is isotype IgG4.

In some embodiments, the bispecific antibody comprises a chimeric hinge that reduces Fcy receptor binding relative to a wild-type hinge of the same isotype.

In some embodiments, the first heavy chain or the second heavy chain of the bispecific antibody, but not both, comprises a CH3 domain comprising a H435R (EU numbering) modification and a Y436F (EU numbering) modification.

In some embodiments, the bispecific antibody comprises a first heavy chain comprising the amino acid sequence of SEQ ID NO: 29. In some embodiments, the bispecific antibody comprises a second heavy chain comprising the amino acid sequence of SEQ ID NO: 31. In some embodiments, the bispecific antibody comprises a first heavy chain comprising the amino acid sequence of SEQ ID NO: 29, a second heavy chain comprising the amino acid sequence of SEQ ID NO: 31, and a common light chain comprising the amino acid sequence of SEQ ID NO: 30.

In some embodiments, the subject has an elevated serum CA-125 level. In some embodiments, the subject has a serum CA-125 level at least two times the upper limit of normal. In some embodiments, the subject has a serum CA-125 level of greater than 92 U/ml.

In some embodiments, the method further comprises administering a second therapeutic agent or therapeutic regimen. In some cases, the second therapeutic agent or therapeutic regimen comprises an anti-PD-1 antibody or antigen-binding fragment thereof. In certain embodiments, the anti-PD-1 antibody is cemiplimab.

In some embodiments, the anti-PD-1 antibody or antigen-binding fragment comprises: (a) three heavy chain complementarity determining regions (HCDR1, HCDR2 and HCDR3) contained within a heavy chain variable region (HCVR) comprising the amino acid sequence of SEQ ID NO: 33; and (b) three light chain complementarity determining regions (LCDR1, LCDR2 and LCDR3) contained within a light chain variable region (LCVR) comprising the amino acid sequence of SEQ ID NO: 34. In some cases, the anti-PD-1 antibody or antigen-binding fragment comprises a HCDR1 comprising the amino acid sequence of SEQ ID NO: 35, a HCDR2 comprising the amino acid sequence of SEQ ID NO: 36, and a HCDR3 comprising the amino acid sequence of SEQ ID NO: 37. In some cases, the anti-PD-1 antibody or antigen-binding fragment comprises a LCDR1 comprising the amino acid sequence of SEQ ID NO: 38, a LCDR2 comprising the amino acid sequence of SEQ ID NO: 39, and a LCDR3 comprising the amino acid sequence of SEQ ID NO: 40. In some cases, the anti-PD-1 antibody or antigen-binding fragment comprises a HCVR comprising the amino acid sequence of SEQ ID NO: 33, and a LCVR comprising the amino acid sequence of SEQ ID NO: 34. In some cases, the anti-PD-1 antibody or antigen-binding fragment is an anti-PD-1 antibody comprising a heavy chain comprising the amino acid sequence of SEQ ID NO: 41 and a light chain comprising the amino acid sequence of SEQ ID NO: 42.

In some embodiments, the bispecific antibody or antigen-binding fragment thereof is administered in a dosing regimen comprising a split initial dose. In some embodiments, the bispecific antibody or antigen-binding fragment thereof is administered to the subject at a dose of from 1 mg to 1000 mg weekly. In some embodiments, the bispecific antibody or antigen-binding fragment thereof is administered to the subject at a dose of from 2 mg to 1000 mg weekly. In some embodiments, the initial dose (e.g., 1 mg or 2 mg) is split into two fractions of equal or non-equal portions. For example, in some cases, the initial dose is split in half (e.g., the initial dose of 1 mg is split into two fractions of 0.5 mg each that are administered on separate days, e.g., consecutive days). In an alternative, the initial dose may be split into non-equal fractions administered on separate days, e.g., consecutive days. In some cases, the bispecific antibody or antigen-binding fragment thereof is administered to the subject at a dose of about 250 mg weekly. In some cases, the 250 mg dose is split into two fractions comprising 50 mg and 200 mg, respectively. In some cases, the bispecific antibody or antigen-binding fragment thereof is administered to the subject at a dose of about 800 mg weekly. In some cases, the 800 mg dose is split into two fractions comprising 50 mg and 750 mg, respectively. In some cases, the bispecific antibody or antigen-binding fragment thereof is administered to the subject at a frequency of once every 3 weeks (e.g., about 250 mg or about 800 mg, or from 10 mg to 1000 mg, once every three weeks). In some cases, the bispecific antibody or antigen-binding fragment is administered at a dose of about 250 mg once every three weeks. In some cases, the 250 mg dose is split into two fractions comprising 50 mg and 200 mg, respectively. In some embodiments, the bispecific antibody is administered to the subject at a dose sufficient to achieve a serum concentration of at least 4 mg/L. In some embodiments, the bispecific antibody is administered in a dosing regimen comprising: (i) administering 1 mg of the bispecific antibody in week 1, optionally wherein the dose is split into a first fraction of about 0.5 mg and a second fraction of about 0.5 mg; (ii) administering 20 mg of the bispecific antibody in week 2, optionally wherein the dose is split into a first fraction of about 10 mg and a second fraction of about 10 mg; and (iii) administering 250 mg of the bispecific antibody in week 3, optionally wherein the dose is split into a first fraction of about 50 mg, and a second fraction of about 200 mg. In some cases, the dosing regimen further comprises administering the bispecific antibody at a dose of about 250 mg once every week from week 4 onwards. In some cases, the dosing regimen further comprises administering the bispecific antibody at a dose of about 250 mg once every three weeks from week 4 onwards. In some embodiments, the bispecific antibody is administered in a dosing regimen comprising: (i) administering 1 mg of the bispecific antibody in week 1; (ii) administering 20 mg of the bispecific antibody in week 2, optionally wherein the dose is split into a first fraction of about 10 mg and a second fraction of about 10 mg; and (iii) administering 800 mg of the bispecific antibody in week 3, optionally wherein the dose is split into a first fraction of about 50 mg, and a second fraction of about 750 mg. In some cases, the dosing regimen further comprises administering the bispecific antibody at a dose of about 800 mg once every week from week 4 onwards. In some cases, the dosing regimen further comprises administering the bispecific antibody at a dose of about 800 mg once every three weeks from week 4 onwards. In some cases, the bispecific antibody or antigen-binding fragment thereof is administered to the subject via intravenous administration. In some cases, the bispecific antibody or antigen-binding fragment thereof is administered to the subject via subcutaneous administration. In some embodiments, the anti-PD-1 antibody or antigen-binding fragment thereof is administered to the subject at a dose of from 300 to 400 mg once every three weeks. In some cases, the anti-PD-1 antibody or antigen-binding fragment thereof is administered to the subject at a dose of 350 mg once every three weeks. In some cases, the anti-PD-1 antibody or antigen-binding fragment is administered intravenously.

In some embodiments of the method, the subject has stable disease, a partial response, or a complete response following administration of the bispecific antibody for at least one week at a dose of 1-800 mg. In some embodiments of the method, the subject has stable disease, a partial response, or a complete response following administration of the bispecific antibody for at least one week at a dose of 20-800 mg.

In some embodiments of the method, the bispecific antibody is administered to the subject at a dose sufficient to achieve a serum concentration of at least 4 mg/L,

In some embodiments of the method, the MIC16 is highly expressed in ≥ 75% of tumor cells in the subject, as determined by immunohistochemical staining. In some embodiments of the method, the subject has: a baseline MUC16 immunohistochemical staining score of 2 in a MUC16-expressing tumor; or a baseline MUC16 immunohistochemical staining score of 2+ in a MUC16-expressing tumor; or a baseline MUC16 immunohistochemical staining score of 3 in a MUC16-expressing tumor; or a baseline MUC16 immunohistochemical staining score of 3+ in a MUC16-expressing tumor; or a baseline MUC16 immunohistochemical staining score of 4 in a MUC16-expressing tumor; or a baseline MUC16 immunohistochemical staining score of 4+ in a MUC16-expressing tumor; or a baseline MUC16 immunohistochemical staining score of 5 in a MUC16-expressing tumor; or a tumor with MUC16-expression in ≥ 50% of tumor cells; or a tumor with MUC16-expression in ≥ 55% of tumor cells; or a tumor with MUC16-expression in ≥ 60% of tumor cells; or a tumor with MUC16-expression in ≥ 65% of tumor cells; or a tumor with MUC16-expression in ≥ 70% of tumor cells; or a tumor with MUC16-expression in ≥ 75% of tumor cells.

In some embodiments of the method, the bispecific antibody is administered intravenously. In some embodiments, the bispecific antibody is administered subcutaneously. In some embodiments of the method, the anti-PD-1 antibody or antigen-binding fragment is administered intravenously.

Other embodiments of the present invention will become apparent from a review of the ensuing detailed description.

### BRIEF DESCRIPTION OF THE FIGURES

FIG. 1 illustrates the binding of various concentrations of anti-MUC16 clone 3A5 and BSMUC16/CD3-001 to CA125, as determined by ELISA (described in Example 2 herein). BSMUC16/CD3-001 and its MUC16 parental antibody displayed a markedly reduced binding signal at all concentrations tested in comparison to an anti-MUC16 clone 3A5 that binds to the repeat region of MUC16.

FIG. 2 illustrates the mean tumor growth curves for groups of mice (5 per group) treated with CD3-binding control + isotype control (Δ), BSMUC16/CD3-005 + isotype control (□), CD3-binding control + anti-PD-1 (A), and BSMUC16/CD3-005 + anti-PD-1 (▪) (as described in Example 3 herein). The combination of an anti-PD-1 antibody and an anti-CD3xMUC16 bispecific antibody synergistically inhibited tumor growth.

FIG. 3 illustrates the impact of T cell incubation with BSMUC16/CD3-001 on the percentage of PD-1 positive T cells.

FIG. 4 illustrates a waterfall plot of monotherapy REGN4018 dose escalation (20-800 mg) showing the best response of various patients.

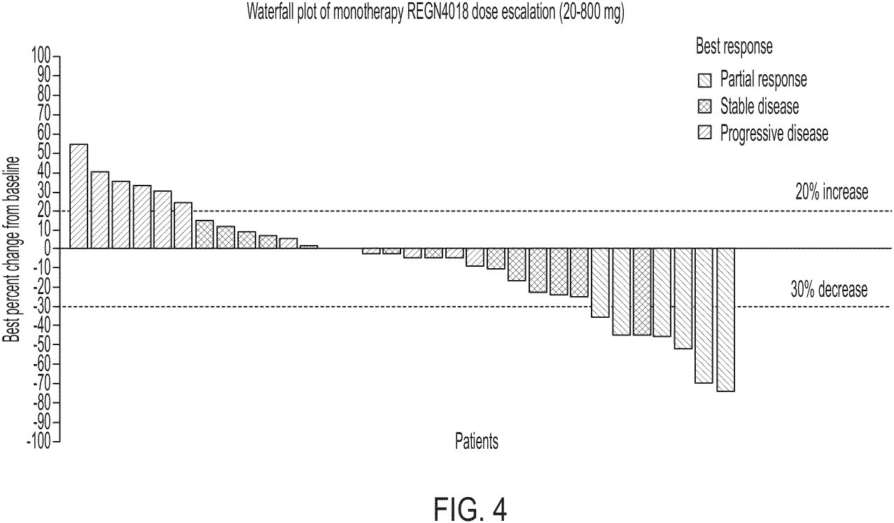

FIG. 5 illustrates an embodiment of a monotherapy dosing regimen for intravenous doses of REGN4018.

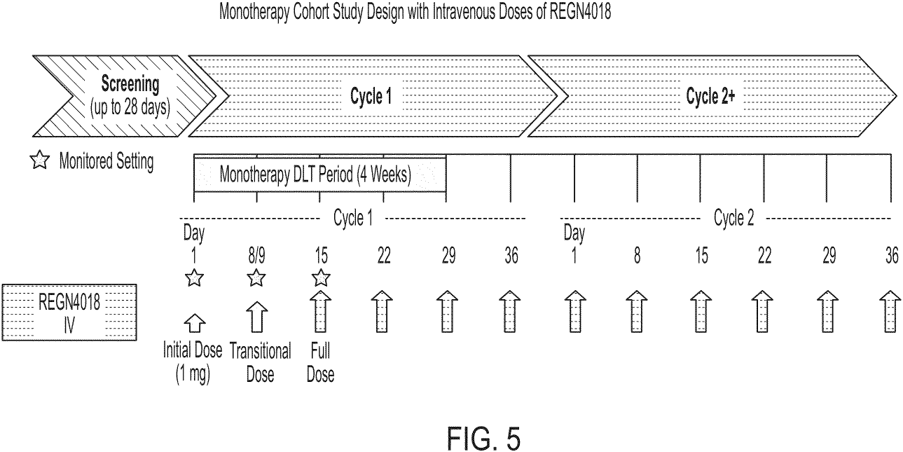

FIG. 6 illustrates an embodiment of a combination therapy dosing regimen for intravenous doses of REGN4018 in combination with cemiplimab.

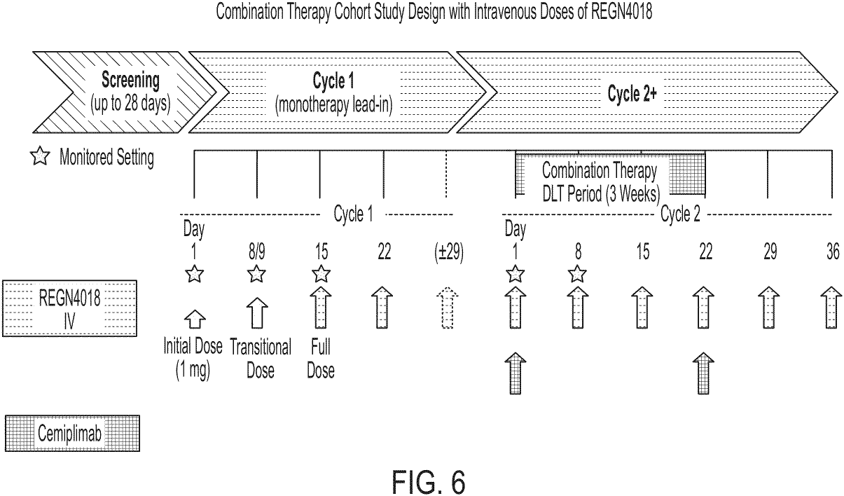

FIG. 7 illustrates an embodiment of a monotherapy dosing regimen with subcutaneous initial and transitional doses of REGN4018. *If the first 3 patients tolerate the second transitional IV dose and subsequent full IV dose of REGN4018 without significant CRS, the second transitional IV dose may be omitted for the remainder of this cohort.

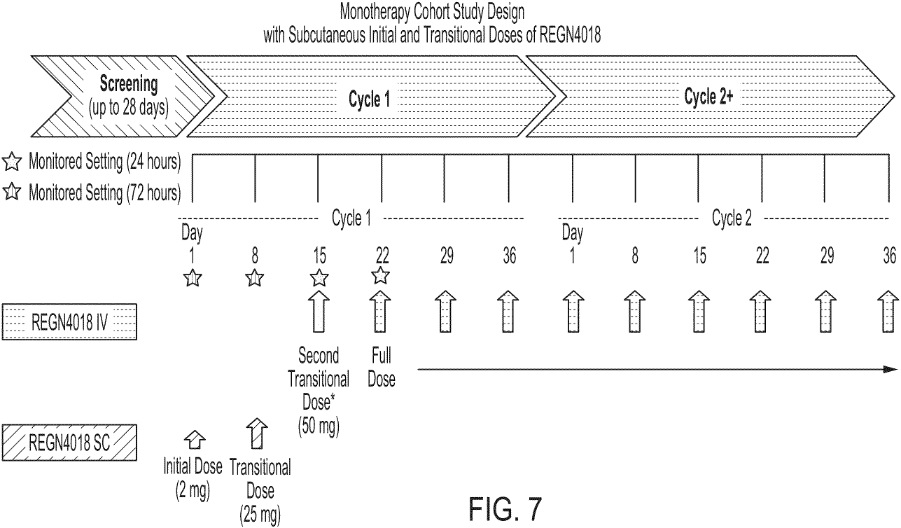

FIG. 8 illustrates an embodiment of a combination therapy dosing regimen with subcutaneous initial and transitional doses of REGN4018 in combination with cemiplimab. *If the first 3 patients tolerate the second transitional IV dose and subsequent full IV dose of REGN4018 without significant CRS, the second transitional IV dose may be omitted for the remainder of this cohort.

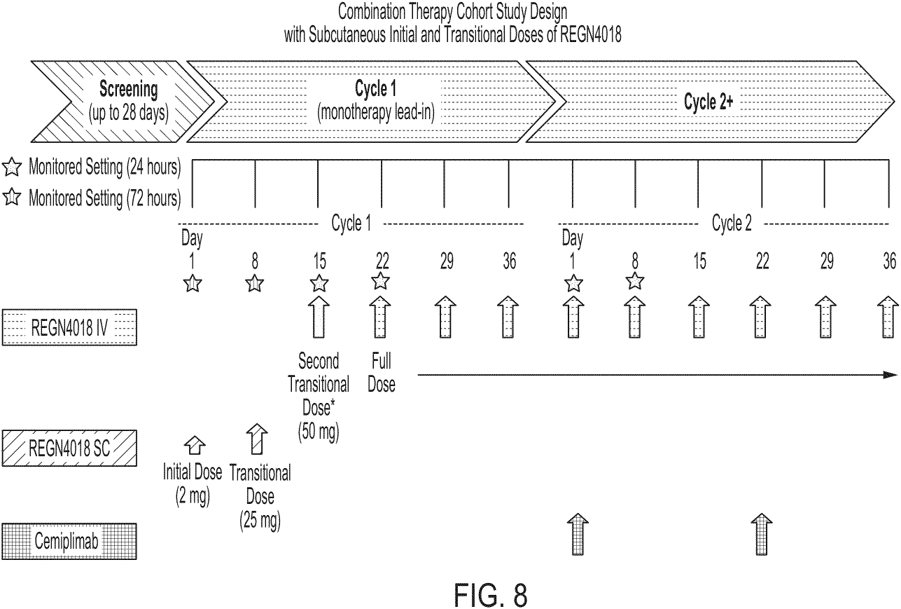

FIG. 9 illustrates an embodiment of a combination therapy dosing regimen with intravenous Q3W doses of REGN4018 in combination with cemiplimab.

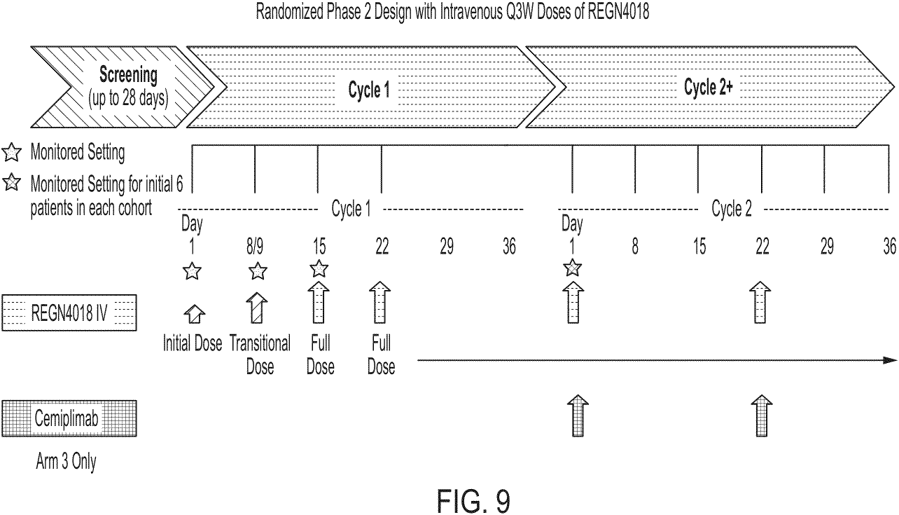

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims. Any embodiments or features of embodiments can be combined with one another, and such combinations are expressly encompassed within the scope of the present invention. Any specific value discussed above or herein may be combined with another related value discussed above or herein to recite a range with the values representing the upper and lower ends of the range, and such ranges are encompassed within the scope of the present disclosure.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present invention, the preferred methods and materials are now described. All patents, applications and non-patent publications mentioned in this specification are incorporated herein by reference in their entireties.

### Methods for Treating or Inhibiting the Growth of Cancers

The present invention includes methods for treating, ameliorating or reducing the severity of at least one symptom or indication, or inhibiting the growth of a cancer (e.g., recurrent ovarian cancer) in a subject. The methods according to this aspect of the invention comprise administering a therapeutically effective amount of a bispecific antibody or antigen-binding fragment thereof against MUC16 and CD3 alone, or in combination with a therapeutically effective amount of an antibody or antigen-binding fragment thereof that specifically binds PD-1 to a subject in need thereof. As used herein, the terms “treat”, “treating”, or the like, mean to alleviate symptoms, eliminate the causation of symptoms either on a temporary or permanent basis, to delay or inhibit tumor growth, to reduce tumor cell load or tumor burden, to promote tumor regression, to cause tumor shrinkage, necrosis and/or disappearance, to prevent tumor recurrence, and/or to increase duration of survival of the subject.

As used herein, the expression “a subject in need thereof” means a human or non-human mammal that exhibits one or more symptoms or indications of cancer, and/or who has been diagnosed with cancer, including an ovarian cancer and who needs treatment for the same. In many embodiments, the term “subject” may be interchangeably used with the term “patient”. For example, a human subject may be diagnosed with a primary or a metastatic tumor and/or with one or more symptoms or indications including, but not limited to, enlarged lymph node(s), swollen abdomen, chest pain/pressure, unexplained weight loss, fever, night sweats, persistent fatigue, loss of appetite, enlargement of spleen, itching. The expression includes subjects with primary or established ovarian tumors. In specific embodiments, the expression includes human subjects that have and need treatment for ovarian cancer or another tumor expressing MUC16, for example, endometrial cancer. In other specific embodiments, the expression includes subjects with MUC16+ tumors (e.g., a tumor with MUC16 expression as determined by flow cytometry or immunohistochemistry). In certain embodiments, the expression includes human subjects with a tumor that shows high expression of MUC16 in >50%, >55%, >60%, >65%, >70% or >75% of tumor cells. The expression of MUC16 may be determined and evaluated by any method known in the art (see e.g., Shimizu et al 2012, Cancer Sci. 103: 739-746). In certain embodiments, the expression includes human subjects with a baseline MUC16 immunohistochemical staining score of 2+ (e.g., 2, 3, 4 or 5) in a MUC16-expressing tumor. In certain embodiments, the expression includes human subjects with a baseline MUC16 immunohistochemical staining score of 2, 2+, 3, 3+, 4, 4+, or 5 in a MUC16-expressing tumor. Immunohistochemical staining scores, in this context, incorporate the percentage of cells, and the intensity and pattern of the staining according to the following standards: score 1 (<5% strong or weak); score 2 (5-50% strong or weak); score 3 (51-75% strong or 51-100% weak); score 4 (76-99% strong); and score 5 (100% strong staining). In certain embodiments, the expression “a subject in need thereof” includes patients with an ovarian cancer that is resistant to or refractory to or is inadequately controlled by prior therapy (e.g., treatment with a conventional anti-cancer agent). For example, the expression includes subjects who have been treated with chemotherapy, such as a platinum-based chemotherapeutic agent (e.g., cisplatin) or a taxol compound (e.g., docetaxel). The expression also includes subjects with an ovarian tumor for which conventional anti-cancer therapy is inadvisable, for example, due to toxic side effects. For example, the expression includes patients who have received one or more cycles of chemotherapy with toxic side effects. In certain embodiments, the expression “a subject in need thereof” includes patients with an ovarian tumor which has been treated but which has subsequently relapsed or metastasized. For example, patients with an ovarian tumor that may have received treatment with one or more anti-cancer agents leading to tumor regression; however, subsequently have relapsed with cancer resistant to the one or more anti-cancer agents (e.g., chemotherapy-resistant cancer) are treated with the methods of the present invention.

The expression “a subject in need thereof” also includes subjects who are at risk of developing ovarian cancer, e.g., persons with a family history of ovarian cancer, persons with a past history of infections associated with ovarian cancer, persons with mutations in the BRCA½ genes, or persons with an immune system compromised due to HIV infection or due to immunosuppressive medications.

In certain embodiments, the methods of the present invention may be used to treat patients that show elevated levels of one or more cancer-associated biomarkers (e.g., programmed death ligand 1 (PD-L1), MUC16, CA125, human epididymis protein 4 (HE4), and/or carcinoembryonic antigen (CEA)). For example, the methods of the present invention comprise administering a therapeutically effective amount of an anti-PD-1 antibody or antigen-binding fragment thereof in combination with a bispecific anti-MUC16/anti-CD3 antibody or antigen-binding fragment thereof to a patient with an elevated level of MUC16 and/or CA125. Methods to determine MUC16 and/or CA125 expression are well-known in the art. In certain embodiments, the expression of MUC16 in tumor tissue is determined by an immunohistochemistry (IHC) assay (see e.g., Bast et al 1981, J. Clin. Invest. 68: 1331-1337). MUC16 expression may be evaluated by any method known in the art (e.g., Shimizu et al 2012, Cancer Sci. 103: 739-746). In certain embodiments, the expression of MUC16 is determined by imaging with a labeled anti-MUC16 antibody, for example, by immuno-positron emission tomography or iPET (described elsewhere herein).

In certain embodiments, the methods of the present invention are used in a subject with an ovarian cancer. The terms “tumor”, “cancer” and “malignancy” are interchangeably used herein. The term “ovarian cancer”, as used herein, refers to tumors of the ovary and fallopian tube, and includes serous cancer, endometrioid carcinoma, clear cell carcinoma, and mucinous carcinoma.

According to certain embodiments, the present invention includes methods for treating, or delaying or inhibiting the growth of a tumor. In certain embodiments, the present invention includes methods to promote tumor regression. In certain embodiments, the present invention includes methods to reduce tumor cell load or to reduce tumor burden. In certain embodiments, the present invention includes methods to prevent tumor recurrence. The methods, according to this aspect of the invention, comprise administering a therapeutically effective amount of a bispecific anti-MUC16/anti-CD3 antibody or antigen-binding fragment thereof alone, or in combination with an anti-PD-1 antibody or antigen-binding fragment thereof to a subject in need thereof, wherein each antibody is administered to the subject in multiple doses, e.g., as part of a specific therapeutic dosing regimen. For example, the therapeutic dosing regimen may comprise administering one or more doses of an anti-MUC16 x CD3 antibody or antigen-binding fragment thereof to the subject at a frequency of about once a day, once every two days, once every three days, once every four days, once every five days, once every six days, once a week, once every two weeks, once every three weeks, once every four weeks, once a month, once every two months, once every three months, once every four months, or less frequently. In certain embodiments, the one or more doses of anti-PD-1 antibody or antigen-binding fragment thereof are administered in combination with one or more doses of a therapeutically effective amount of a bispecific anti-MUC16/anti-CD3 antibody or antigen-binding fragment thereof, wherein the one or more doses of the anti-PD-1 antibody or antigen-binding fragment thereof are administered to the subject at a frequency of about once a day, once every two days, once every three days, once every four days, once every five days, once every six days, once a week, once every two weeks, once every three weeks, once every four weeks, once a month, once every two months, once every three months, once every four months, or less frequently.

In certain embodiments, each dose of the anti-MUC16/anti-CD3 antibody or antigen-binding fragment thereof is administered in more than 1 fractions, e.g., in 2-5 fractions (“split dosing”) within the given dosing period. The anti-MUC16/anti-CD3 bispecific antibody or antigen-binding fragment thereof may be administered in split doses to reduce or eliminate the cytokine “spikes” induced in response to administration of the antibody. Cytokine spikes refer to the clinical symptoms of the cytokine release syndrome (“cytokine storm”) and infusion related reactions. In certain embodiments, the methods of the present invention comprise administering one or more doses of anti-PD-1 antibody or antigen-binding fragment thereof in combination with one or more doses of a bispecific anti-MUC16/anti-CD3 antibody or antigen-binding fragment thereof to a subject in need thereof, wherein a dose of the bispecific antibody or antigen-binding fragment thereof is administered as split doses, or in more than 1 fractions, e.g., as 2 fractions, as 3 fractions, as 4 fractions or as 5 fractions within the given dosing period. In certain embodiments, a dose of the bispecific antibody or antigen-binding fragment thereof is split into 2 or more fractions, wherein each fraction comprises an amount of the antibody or antigen-binding fragment thereof equal to the other fractions. For example, a dose of anti-MUC16/anti-CD3 antibody or antigen-binding fragment thereof comprising 1000 micrograms may be administered once a week, wherein the dose is administered in 2 fractions within the week, each fraction comprising 500 micrograms. In certain embodiments, a dose of the bispecific antibody or antigen-binding fragment thereof is administered split into 2 or more fractions, wherein the fractions comprise unequal amounts of the antibody, e.g., more than or less than the first fraction. For example, a dose of anti-MUC16/anti-CD3 antibody or antigen-binding fragment thereof comprising 1000 micrograms may be administered once a week, wherein the dose is administered in 2 fractions within the week, wherein the first fraction comprises 700 micrograms and the second fraction comprises 300 micrograms. As another example, a dose of anti-MUC16/anti-CD3 antibody or antigen-binding fragment thereof comprising 1000 micrograms may be administered once in 2 weeks, wherein the dose is administered in 3 fractions within the 2-week period, wherein the first fraction comprises 400 micrograms, the second fraction comprises 300 micrograms and the third fraction comprises 300 micrograms.

In certain embodiments, the present invention provides a method of treating a MUC16-expressing cancer (e.g., ovarian cancer, fallopian tube cancer, or primary peritoneal cancer, including cancers refractory to multiple rounds of prior therapy as discussed herein) in a subject in need thereof, comprising administering to the subject a bispecific antibody comprising a first antigen-binding domain that specifically binds mucin 16 (MUC16) on a target tumor cell, and a second antigen-binding domain that specifically binds human CD3 on a T cell, wherein the bispecific antibody is administered in a dosing regimen comprising: (i) administering 1 mg of the bispecific antibody in week 1, optionally wherein the dose is split into a first fraction of about 0.5 mg and a second fraction of about 0.5 mg; (ii) administering 20 mg of the bispecific antibody in week 2, optionally wherein the dose is split into a first fraction of about 10 mg and a second fraction of about 10 mg; and (iii) administering 250 mg of the bispecific antibody in week 3, optionally wherein the dose is split into a first fraction of about 50 mg, and a second fraction of about 200 mg. In some cases, the method further comprises administering the bispecific antibody at a dose of about 250 mg once every week from week 4 onwards. In some cases, the method further comprises administering the bispecific antibody at a dose of about 250 mg once every three weeks from week 4 onwards. In some cases, the method further comprises administering the bispecific antibody at a dose of about 800 mg once every three weeks from week 4 onwards. In some cases, the method further comprises administering an anti-PD-1 antibody to the subject at a dose of from 300 to 400 mg (e.g., 350 mg) once every three weeks.

In certain embodiments, the present invention includes methods to inhibit, retard or stop tumor metastasis or tumor infiltration into peripheral organs. The methods, according to this aspect, comprise administering a therapeutically effective amount of a bispecific anti-MUC16/anti-CD3 antibody or antigen-binding fragment thereof alone, or in combination with an anti-PD-1 antibody or antigen-binding fragment thereof to a subject in need thereof.

In specific embodiments, the present invention provides methods for increased antitumor efficacy or increased tumor inhibition. The methods, according to this aspect of the invention, comprise administering to a subject with an ovarian cancer a therapeutically effective amount of an anti-PD-1 antibody or antigen-binding fragment thereof prior to administering a therapeutically effective amount of a bispecific anti-MUC16/anti-CD3 antibody or antigen-binding fragment thereof, wherein the anti-PD-1 antibody or antigen-binding fragment thereof may be administered about 1 day, more than 1 day, more than 2 days, more than 3 days, more than 4 days, more than 5 days, more than 6 days, more than 7 days, or more than 8 days prior to the bispecific antibody or antigen-binding fragment thereof. In certain embodiments, the methods provide for increased tumor inhibition, e.g., by about 20%, more than 20%, more than 30%, more than 40% more than 50%, more than 60%, more than 70% or more than 80% as compared to a subject administered with the bispecific antibody or antigen-binding fragment thereof prior to the anti-PD-1 antibody or antigen-binding fragment thereof.

In certain embodiments, the methods of the present invention comprise administering a therapeutically effective amount of a bispecific anti-CD3xMUC16 antibody or antigen-binding fragment thereof alone, or in combination with an anti-PD-1 antibody or antigen-binding fragment thereof to a subject with an ovarian cancer. In specific embodiments, the ovarian cancer is serous cancer. In further embodiments, the ovarian cancer is indolent or aggressive. In certain embodiments, the subject is not responsive to prior therapy or has relapsed after prior therapy (e.g., platinum-based therapy). In some embodiments, the subject has a CA-125 level that is equal to or greater than 2 times the upper limit of normal (ULN) (e.g., equal to or greater than about 60 U/ml). In various embodiments, the subject’s serum CA-125 level (prior to treatment) is at or greater than 60, 65, 70, 75, 80, 85, 90, 95, 100, 110, 120, 130, 140, 150, 160, 170, 180, 190, 200, 250, 300, 350, 400, 450, 500, 550, 600, 650 or 700 U/ml. In certain embodiments, the methods of the present invention further comprise administering an additional therapeutic agent to the subject.

In certain embodiments, the methods of the present invention comprise administering a therapeutically effective amount of a bispecific anti-MUC16/anti-CD3 antibody or antigen-binding fragment thereof to a subject with a MUC16+ cancer. In specific embodiments, the cancer is an ovarian cancer. In further embodiments, the ovarian cancer is indolent or aggressive. In some embodiments, the cancer is a platinum-resistant ovarian cancer. In some embodiments, the cancer is a taxol-resistant ovarian cancer. In some embodiments, the cancer is fallopian tube cancer. In some embodiments, the cancer is primary peritoneal cancer, optionally in which the patient has elevated levels of serum CA-125 (e.g., at least 2x ULN). In certain embodiments, the subject is not responsive to prior therapy or has relapsed after prior therapy (e.g., chemotherapy).

In certain embodiments, the methods of the present invention comprise administering an anti-PD-1 antibody or antigen-binding fragment thereof in combination with a bispecific anti-MUC16/anti-CD3 antibody or antigen-binding fragment thereof to a subject in need thereof as a “first line” treatment (e.g., initial treatment). In other embodiments, an anti-PD-1 antibody or antigen-binding fragment thereof in combination with a bispecific anti-MUC16/anti-CD3 antibody or antigen-binding fragment thereof is administered as a “second line” treatment (e.g., after prior therapy). For example, an anti-PD-1 antibody or antigen-binding fragment thereof in combination with a bispecific anti-MUC16/anti-CD3 antibody or antigen-binding fragment thereof is administered as a “second line” treatment to a subject that has relapsed after prior therapy with, e.g., chemotherapy (e.g., platinum-based chemotherapy).

In certain embodiments, the methods of the present invention are used to treat a patient with a MRD-positive disease. Minimum residual disease (MRD) refers to small numbers of cancer cells that remain in the patient during or after treatment, wherein the patient may or may not show symptoms or signs of the disease. Such residual cancer cells, if not eliminated, frequently lead to relapse of the disease. The present invention includes methods to inhibit and/or eliminate residual cancer cells in a patient upon MRD testing. MRD may be assayed according to methods known in the art (e.g., MRD flow cytometry). The methods, according to this aspect of the invention, comprise administering a bispecific anti-MUC16/anti-CD3 antibody or antigen-binding fragment thereof alone, or in combination with an anti-PD-1 antibody or antigen-binding fragment thereof to a subject in need thereof.

The methods of the present invention, according to certain embodiments, comprise administering to a subject a therapeutically effective amount of a bispecific anti-MUC16/anti-CD3 antibody or antigen-binding fragment thereof alone, or in combination with an anti-PD-1 antibody or antigen-binding fragment thereof and, optionally, a third therapeutic agent. The third therapeutic agent may be an agent selected from the group consisting of, e.g., radiation, chemotherapy, surgery, a cancer vaccine, a PD-L1 inhibitor (e.g., an anti-PD-L1 antibody), a LAG3 inhibitor (e.g., an anti-LAG3 antibody), a CTLA-4 inhibitor (e.g., an anti-CTLA-4 antibody), a TIM3 inhibitor, a BTLA inhibitor, a TIGIT inhibitor, a CD47 inhibitor, an indoleamine-2,3-dioxygenase (IDO) inhibitor, a vascular endothelial growth factor (VEGF) antagonist, an Ang2 inhibitor, a transforming growth factor beta (TGF.beta.) inhibitor, an epidermal growth factor receptor (EGFR) inhibitor, an antibody to a tumor-specific antigen (e.g., CA9, CA125, melanoma-associated antigen 3 (MAGE3), carcinoembryonic antigen (CEA), vimentin, tumor-M2-PK, prostate-specific antigen (PSA), mucin-1, MART-1, and CA19-9), a vaccine (e.g., Bacillus Calmette-Guerin), granulocyte-macrophage colony-stimulating factor, a cytotoxin, a chemotherapeutic agent, an IL-6R inhibitor, an IL-4R inhibitor, an IL-10 inhibitor, a cytokine such as IL-2, IL-7, IL-21, and IL-15, an anti-inflammatory drug such as corticosteroids, and non-steroidal anti-inflammatory drugs, and a dietary supplement such as anti-oxidants. In certain embodiments, the antibodies may be administered in combination with therapy including a chemotherapeutic agent (e.g., paclitaxel, carboplatin, doxorubicin, cyclophosphamide, cisplatin, gemcitabine or docetaxel), radiation and surgery. As used herein, the phrase “in combination with” means that the antibodies are administered to the subject at the same time as, just before, or just after administration of the third therapeutic agent. In certain embodiments, the third therapeutic agent is administered as a co-formulation with the antibodies.

In certain embodiments, the methods of the present invention comprise administering to a subject in need thereof a therapeutically effective amount of a bispecific anti-MUC16/anti-CD3 antibody or antigen-binding fragment thereof alone, or in combination with an anti-PD-1 antibody or antigen-binding fragment thereof. Where the combination is administered, the administration of the antibodies (or fragments) leads to increased inhibition of tumor growth. In certain embodiments, tumor growth is inhibited by at least about 10%, about 20%, about 30%, about 40%, about 50%, about 60%, about 70% or about 80% as compared to an untreated subject or a subject administered with either antibody (or fragment) as monotherapy. In certain embodiments, the administration of an anti-PD-1 antibody or antigen-binding fragment thereof and a bispecific anti-MUC16/anti-CD3 antibody or antigen-binding fragment thereof leads to increased tumor regression, tumor shrinkage and/or disappearance. In certain embodiments, the administration of an anti-PD-1 antibody or antigen-binding fragment thereof and a bispecific anti-MUC16/anti-CD3 antibody or antigen-binding fragment thereof leads to delay in tumor growth and development, e.g., tumor growth may be delayed by about 3 days, more than 3 days, about 7 days, more than 7 days, more than 15 days, more than 1 month, more than 3 months, more than 6 months, more than 1 year, more than 2 years, or more than 3 years as compared to an untreated subject or a subject treated with either antibody (or fragment) as monotherapy. In certain embodiments, administration of an anti-PD-1 antibody or antigen-binding fragment thereof in combination with a bispecific anti-MUC16/anti-CD3 antibody or antigen-binding fragment thereof prevents tumor recurrence and/or increases duration of survival of the subject, e.g., increases duration of survival by more than 15 days, more than 1 month, more than 3 months, more than 6 months, more than 12 months, more than 18 months, more than 24 months, more than 36 months, or more than 48 months than an untreated subject or a subject which is administered either antibody (or fragment) as monotherapy. In certain embodiments, administration of the antibodies in combination increases progression-free survival or overall survival. In certain embodiments, administration of an anti-PD-1 antibody or antigen-binding fragment thereof in combination with a bispecific anti-MUC16/anti-CD3 antibody or antigen-binding fragment thereof increases response and duration of response in a subject, e.g., by more than 2%, more than 3%, more than 4%, more than 5%, more than 6%, more than 7%, more than 8%, more than 9%, more than 10%, more than 20%, more than 30%, more than 40% or more than 50% over an untreated subject or a subject which has received either antibody (or fragment) as monotherapy. In certain embodiments, administration of an anti-PD-1 antibody or antigen-binding fragment thereof and a bispecific anti-MUC16/anti-CD3 antibody or antigen-binding fragment thereof to a subject with an ovarian cancer leads to complete disappearance of all evidence of tumor cells (“complete response”). In certain embodiments, administration of an anti-PD-1 antibody or antigen-binding fragment thereof and a bispecific anti-MUC16/anti-CD3 antibody or antigen-binding fragment thereof to a subject with an ovarian cancer leads to at least 30% or more decrease in tumor cells or tumor size (“partial response”). In certain embodiments, administration of an anti-PD-1 antibody or antigen-binding fragment thereof and a bispecific anti-MUC16/anti-CD3 antibody or antigen-binding fragment thereof to a subject with an ovarian cancer leads to complete or partial disappearance of tumor cells/lesions including new measurable lesions. Tumor reduction can be measured by any of the methods known in the art, e.g., X-rays, positron emission tomography (PET), computed tomography (CT), magnetic resonance imaging (MRI), cytology, histology, or molecular genetic analyses. In certain embodiments, administration of an anti-PD-1 antibody or antigen-binding fragment thereof and a bispecific anti-MUC16/anti-CD3 antibody or antigen-binding fragment thereof produces a synergistic anti-tumor effect that exceeds the combined effects of the two agents when administered alone.

In certain embodiments, the combination of administered antibodies (or fragments) is safe and well-tolerated by a patient wherein there is no increase in an adverse side effect (e.g., increased cytokine release (“cytokine storm”) or increased T-cell activation) as compared to a patient administered with the bispecific antibody (or fragment) as monotherapy.

In certain cases, the response of a subject to therapy is categorized as a complete response (CR), a partial response (PR), progressive disease (PD), or as stable disease (SD). A CR is defined as disappearance of all target lesions, and a reduction in short axis of any pathological lymph nodes (whether target or non-target) to <10 mm (<1 cm). A PR is defined as an at least 30% decrease in the sum of the diameters of target lesions, taking as reference the baseline sum diameters. PD is defined as an at least 20% increase in the sum of the diameters of target lesions, taking as reference the smallest sum on study (this includes the baseline sum if that is the smallest on study). In addition to the relative increase of 20%, the sum must also demonstrate an absolute increase of at least 5 mm (0.5 cm). (Note: the appearance of one or more new lesions is also considered a progression). SD is defined as neither sufficient shrinkage to qualify for PR nor sufficient increase to qualify for PD, taking as reference the smallest sum diameters while on study.

### Anti-PD-1 Antibodies and Antigen-Binding Fragments Thereof

According to certain exemplary embodiments of the present invention, the methods comprise administering a therapeutically effective amount of an anti-PD-1 antibody or antigen-binding fragment thereof. The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or V_{H}) and a heavy chain constant region. The heavy chain constant region comprises three domains, C_{H}1, C_{H}2 and CH3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of the anti-IL-4R antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homodimer or hetero-dimer (or other multimer) of any of the variable and constant domain configurations listed above in non-covalent association with one another and/or with one or more monomeric V_{H} or V_{L} domain (e.g., by disulfide bond(s)).

The term “antibody,” as used herein, also includes multispecific (e.g., bispecific) antibodies. A multispecific antibody or antigen-binding fragment of an antibody will typically comprise at least two different variable domains, wherein each variable domain is capable of specifically binding to a separate antigen or to a different epitope on the same antigen. Any multispecific antibody format may be adapted for use in the context of an antibody or antigen-binding fragment of an antibody of the present invention using routine techniques available in the art. For example, the present invention includes methods comprising the use of bispecific antibodies wherein one arm of an immunoglobulin is specific for PD-1 or a fragment thereof, and the other arm of the immunoglobulin is specific for a second therapeutic target or is conjugated to a therapeutic moiety. Exemplary bispecific formats that can be used in the context of the present invention include, without limitation, e.g., scFv-based or diabody bispecific formats, IgG-scFv fusions, dual variable domain (DVD)-lg, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab.sup.2 bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc. [Epub: Dec. 4, 2012]).

The antibodies used in the methods of the present invention may be human antibodies. The term “human antibody,” as used herein, is intended to include antibodies having variable and constant regions derived from human germline immunoglobulin sequences. The human antibodies of the invention may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods of the present invention may be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

According to certain embodiments, the antibodies used in the methods of the present invention specifically bind PD-1. The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” PD-1, as used in the context of the present invention, includes antibodies that bind PD-1 or portion thereof with a K_{D} of less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human PD-1 may, however, have cross-reactivity to other antigens, such as PD-1 molecules from other (non-human) species.

According to certain exemplary embodiments of the present invention, the anti-PD-1 antibody, or antigen-binding fragment thereof comprises a heavy chain variable region (HCVR), light chain variable region (LCVR), and/or complementarity determining regions (CDRs) comprising any of the amino acid sequences of the anti-PD-1 antibodies as set forth in US Pat. No. 9,987,500. In certain exemplary embodiments, the anti-PD-1 antibody or antigen-binding fragment thereof that can be used in the context of the methods of the present invention comprises the heavy chain complementarity determining regions (HCDRs) of a heavy chain variable region (HCVR) comprising the amino acid sequence of SEQ ID NO: 33 and the light chain complementarity determining regions (LCDRs) of a light chain variable region (LCVR) comprising the amino acid sequence of SEQ ID NO: 34. According to certain embodiments, the anti-PD-1 antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO: 35; the HCDR2 comprises the amino acid sequence of SEQ ID NO: 36; the HCDR3 comprises the amino acid sequence of SEQ ID NO: 37; the LCDR1 comprises the amino acid sequence of SEQ ID NO: 38; the LCDR2 comprises the amino acid sequence of SEQ ID NO: 39; and the LCDR3 comprises the amino acid sequence of SEQ ID NO: 40. In yet other embodiments, the anti-PD-1 antibody or antigen-binding fragment thereof comprises an HCVR comprising SEQ ID NO: 33 and an LCVR comprising SEQ ID NO: 34. In certain embodiments, the methods of the present invention comprise the use of an anti-PD-1 antibody, wherein the antibody comprises a heavy chain comprising the amino acid sequence of SEQ ID NO: 41. In some embodiments, the anti-PD-1 antibody comprises a light chain comprising the amino acid sequence of SEQ ID NO: 42. An exemplary antibody comprising a HCVR comprising the amino acid sequence of SEQ ID NO: 33 and a LCVR comprising the amino acid sequence of SEQ ID NO: 34 is the fully human anti-PD-1 antibody known as REGN2810 (also known as cemiplimab, LIBTAYO®). According to certain exemplary embodiments, the methods of the present invention comprise the use of REGN2810, or a bioequivalent thereof. The term “bioequivalent”, as used herein, refers to anti-PD-1 antibodies or PD-1-binding proteins or fragments thereof that are pharmaceutical equivalents or pharmaceutical alternatives whose rate and/or extent of absorption do not show a significant difference with that of REGN2810 when administered at the same molar dose under similar experimental conditions, either single dose or multiple dose. In the context of the invention, the term refers to antigen-binding proteins that bind to PD-1 which do not have clinically meaningful differences with REGN2810 in their safety, purity and/or potency.

Other anti-PD-1 antibodies that can be used in the context of the methods of the present invention include, e.g., the antibodies referred to and known in the art as nivolumab (U.S. Pat. No. 8,008,449), pembrolizumab (U.S. Pat. No. 8,354,509), MEDI0608 (U.S. Pat. No. 8,609,089), pidilizumab (U.S. Pat. No. 8,686,119), or any of the anti-PD-1 antibodies as set forth in U.S. Pat. Nos. 6,808,710, 7,488,802, 8,168,757, 8,354,509, 8,779,105, or 8,900,587.

The anti-PD-1 antibodies used in the context of the methods of the present invention may have pH-dependent binding characteristics. For example, an anti-PD-1 antibody for use in the methods of the present invention may exhibit reduced binding to PD-1 at acidic pH as compared to neutral pH. Alternatively, an anti-PD-1 antibody of the invention may exhibit enhanced binding to its antigen at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding to PD-1 at acidic pH as compared to neutral pH” is expressed in terms of a ratio of the K_{D} value of the antibody binding to PD-1 at acidic pH to the K_{D} value of the antibody binding to PD-1 at neutral pH (or vice versa). For example, an antibody or antigen-binding fragment thereof may be regarded as exhibiting “reduced binding to PD-1 at acidic pH as compared to neutral pH” for purposes of the present invention if the antibody or antigen-binding fragment thereof exhibits an acidic/neutral K_{D} ratio of about 3.0 or greater. In certain exemplary embodiments, the acidic/neutral K_{D} ratio for an antibody or antigen-binding fragment of the present invention can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0, 25.0, 30.0, 40.0, 50.0, 60.0, 70.0, 100.0, or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained. As used herein, the expression “acidic pH” means a pH of 6.0 or less.

### Bispecific Anti-MUC16/Anti-CD3 Antibodies and Antigen-Binding Fragments Thereof

According to certain exemplary embodiments of the present invention, the methods comprise administering a therapeutically effective amount of a bispecific antibody or antigen-binding fragment thereof that specifically binds CD3 and MUC16. Such antibodies and fragments may be referred to herein as, e.g., “anti-MUC16/anti-CD3,” or “anti-MUC16xCD3” or “MUC16xCD3” bispecific antibodies or antigen-binding fragments thereof, or other similar terminology.

As used herein, the expression “bispecific antibody” refers to an immunoglobulin protein comprising at least a first antigen-binding domain and a second antigen-binding domain. In the context of the present invention, the first antigen-binding domain specifically binds a first antigen (e.g., MUC16), and the second antigen-binding domain specifically binds a second, distinct antigen (e.g., CD3). Each antigen-binding domain of a bispecific antibody comprises a heavy chain variable domain (HCVR) and a light chain variable domain (LCVR), each comprising three CDRs. In the context of a bispecific antibody, the CDRs of the first antigen-binding domain may be designated with the prefix “A” and the CDRs of the second antigen-binding domain may be designated with the prefix “B”. Thus, the CDRs of the first antigen-binding domain may be referred to herein as A-HCDR1, A-HCDR2, and A-HCDR3; and the CDRs of the second antigen-binding domain may be referred to herein as B-HCDR1, B-HCDR2, and B-HCDR3.

The first antigen-binding domain and the second antigen-binding domain are each connected to a separate multimerizing domain. As used herein, a “multimerizing domain” is any macromolecule, protein, polypeptide, peptide, or amino acid that has the ability to associate with a second multimerizing domain of the same or similar structure or constitution. In the context of the present invention, the multimerizing component is an Fc portion of an immunoglobulin (comprising a C_{H2}-C_{H3} domain), e.g., an Fc domain of an IgG selected from the isotypes IgG1, IgG2, IgG3, and IgG4, as well as any allotype within each isotype group.

Bispecific antibodies of the present invention typically comprise two multimerizing domains, e.g., two Fc domains that are each individually part of a separate antibody heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

Any bispecific antibody format or technology may be used to make the bispecific antigen-binding molecules of the present invention. For example, an antibody or fragment thereof having a first antigen binding specificity can be functionally linked (e.g., by chemical coupling, genetic fusion, noncovalent association or otherwise) to one or more other molecular entities, such as another antibody or antibody fragment having a second antigen-binding specificity to produce a bispecific antigen-binding molecule. Specific exemplary bispecific formats that can be used in the context of the present invention include, without limitation, e.g., scFv-based or diabody bispecific formats, IgG-scFv fusions, dual variable domain (DVD)-lg, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mab₂ bispecific formats (see, e.g., Klein et al. 2012, mAbs 4:6, 1-11, and references cited therein, for a review of the foregoing formats).

In the context of bispecific antibodies of the present invention, Fc domains may comprise one or more amino acid changes (e.g., insertions, deletions or substitutions) as compared to the wild-type, naturally occurring version of the Fc domain. For example, the invention includes bispecific antigen-binding molecules comprising one or more modifications in the Fc domain that results in a modified Fc domain having a modified binding interaction (e.g., enhanced or diminished) between Fc and FcRn. In one embodiment, the bispecific antigen-binding molecule comprises a modification in a C_{H}2 or a C_{H}3 region, wherein the modification increases the affinity of the Fc domain to FcRn in an acidic environment (e.g., in an endosome where pH ranges from about 5.5 to about 6.0). Non-limiting examples of such Fc modifications are disclosed in US Pat. Publication No. 20150266966, incorporated herein in its entirety.

The present invention also includes bispecific antigen-binding molecules comprising a first CH3 domain and a second Ig CH3 domain, wherein the first and second Ig CH3 domains differ from one another by at least one amino acid, and wherein at least one amino acid difference reduces binding of the bispecific antibody to Protein A as compared to a bi-specific antibody lacking the amino acid difference. In one embodiment, the first Ig CH3 domain binds Protein A and the second Ig CH3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). See, for example, U.S. Pat. No. 8,586,713. Further modifications that may be found within the second C_{H}3 include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E419Q, and V422I by EU) in the case of IgG4 antibodies.

In certain embodiments, the Fc domain may be chimeric, combining Fc sequences derived from more than one immunoglobulin isotype. For example, a chimeric Fc domain can comprise part or all of a CH2 sequence derived from a human IgG1, human IgG2 or human IgG4 C_{H}2 region, and part or all of a C_{H}3 sequence derived from a human IgG1, human IgG2 or human IgG4. A chimeric Fc domain can also contain a chimeric hinge region. For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region. A particular example of a chimeric Fc domain that can be included in any of the antigen-binding molecules set forth herein comprises, from N- to C-terminus: [IgG4 C_{H}1]-[IgG4 upper hinge]-[IgG2 lower hinge]-[IgG4 CH2]-[lgG4 CH3]. Another example of a chimeric Fc domain that can be included in any of the antigen-binding molecules set forth herein comprises, from N- to C-terminus: [IgG1 C_{H}1]-[IgG1 upper hinge]-[IgG2 lower hinge]-[lgG4 C_{H}2]-[IgG1 CH3]. These and other examples of chimeric Fc domains that can be included in any of the antigen-binding molecules of the present invention are described in US Pat. Publication No. 20140243504, which is herein incorporated in its entirety. Chimeric Fc domains having these general structural arrangements, and variants thereof, can have altered Fc receptor binding, which in turn affects Fc effector function.

According to certain exemplary embodiments of the present invention, the bispecific anti-MUC16/anti-CD3 antibody, or antigen-binding fragment thereof comprises heavy chain variable regions (A-HCVR and B-HCVR), light chain variable regions (A-LCVR and B-LCVR), and/or complementarity determining regions (CDRs) comprising any of the amino acid sequences of the bispecific anti-MUC16/anti-CD3 antibodies as set forth in US Pat. Publication No. 20180112001. In certain exemplary embodiments, the bispecific anti-MUC16/anti-CD3 antibody or antigen-binding fragment thereof that can be used in the context of the methods of the present invention comprises: (a) a first antigen-binding arm comprising the heavy chain complementarity determining regions (A-HCDR1, A-HCDR2 and A-HCDR3) of a heavy chain variable region (A-HCVR) comprising the amino acid sequence of SEQ ID NO: 1 and the light chain complementarity determining regions (A-LCDR1, A-LCDR2 and A-LCDR3) of a light chain variable region (A-LCVR) comprising the amino acid sequence of SEQ ID NO: 2; and (b) a second antigen-binding arm comprising the heavy chain CDRs (B-HCDR1, B-HCDR2 and B-HCDR3) of a HCVR (B-HCVR) comprising an amino acid sequence of SEQ ID NO: 3, SEQ ID NO: 4, SEQ ID NO: 5, SEQ ID NO: 6 or SEQ ID NO: 7, and the light chain CDRs (B-LCDR1, B-LCDR2 and B-LCDR3) of a LCVR (B-LCVR) comprising the amino acid sequence of SEQ ID NO: 2. According to certain embodiments, the A-HCDR1 comprises the amino acid sequence of SEQ ID NO: 8; the A-HCDR2 comprises the amino acid sequence of SEQ ID NO: 9; the A-HCDR3 comprises the amino acid sequence of SEQ ID NO: 10; the A-LCDR1 comprises the amino acid sequence of SEQ ID NO: 11; the A-LCDR2 comprises the amino acid sequence of SEQ ID NO: 12; the A-LCDR3 comprises the amino acid sequence of SEQ ID NO: 13; the B-HCDR1 comprises the amino acid sequence of SEQ ID NO: 14, SEQ ID NO: 17, SEQ ID NO: 20, SEQ ID NO: 23, or SEQ ID NO: 26; the B-HCDR2 comprises the amino acid sequence of SEQ ID NO: 15, SEQ ID NO: 18, SEQ ID NO: 21, SEQ ID NO: 24, or SEQ ID NO: 27; and the B-HCDR3 comprises the amino acid sequence of SEQ ID NO: 16, SEQ ID NO: 19, SEQ ID NO: 22, SEQ ID NO: 25, or SEQ ID NO: 28; and the B-LCDR1 comprises the amino acid sequence of SEQ ID NO: 11; the B-LCDR2 comprises the amino acid sequence of SEQ ID NO: 12; the B-LCDR3 comprises the amino acid sequence of SEQ ID NO: 13. In yet other embodiments, the bispecific anti-MUC16/anti-CD3 antibody or antigen-binding fragment thereof comprises: (a) a first antigen-binding arm comprising a HCVR (A-HCVR) comprising SEQ ID NO: 1 and a LCVR (A-LCVR) comprising SEQ ID NO: 2; and (b) a second antigen-binding arm comprising a HCVR (B-HCVR) comprising SEQ ID NO: 3, SEQ ID NO: 4, SEQ ID NO: 5, SEQ ID NO: 6, or SEQ ID NO: 7, and a LCVR (B-LCVR) comprising SEQ ID NO: 2. In certain exemplary embodiments, the bispecific anti-CD3xMUC16 antibody comprises a MUC16-binding arm comprising a heavy chain comprising the amino acid sequence of SEQ ID NO: 29 and a light chain comprising the amino acid sequence of SEQ ID NO: 30, and a CD3-binding arm comprising a heavy chain comprising the amino acid sequence of SEQ ID NO: 31 and a light chain comprising the amino acid sequence of SEQ ID NO: 30. In certain exemplary embodiments, the bispecific anti-CD3xMUC16 antibody comprises a MUC16-binding arm comprising a heavy chain comprising the amino acid sequence of SEQ ID NO: 29 and a light chain comprising the amino acid sequence of SEQ ID NO: 30, and a CD3-binding arm comprising a heavy chain comprising the amino acid sequence of SEQ ID NO: 32 and a light chain comprising the amino acid sequence of SEQ ID NO: 30.

In certain embodiments, the anti-tumor activity of the bispecific anti-CD3xMUC16 antibodies or antigen-binding fragments thereof of the present invention is not substantially impeded by the presence of high levels (e.g., up to 10,000 U/ml) of circulating CA125. Serum levels of CA125 are increased in the serum of the majority of ovarian cancer patients (median published levels are about 656 U/ml). As demonstrated in Example 2, below, high levels of CA125 in serum or ascites will not significantly interfere with the anti-tumor profile of the bispecific antibodies of the present invention.

Other bispecific anti-MUC16/anti-CD3 antibodies that can be used in the context of the methods of the present invention include, e.g., any of the antibodies as set forth in US Pat. Publication No. 20180112001.

### Combination Therapies

The methods of the present invention, according to certain embodiments, comprise administering to the subject an anti-MUC16/anti-CD3 bispecific antibody or antigen-binding fragment thereof in combination with an anti-PD-1 antibody or antigen-binding fragment thereof. In certain embodiments, the methods of the present invention comprise administering the antibodies (or fragments) for additive or synergistic activity to treat cancer, preferably an ovarian cancer. As used herein, the expression “in combination with” means that the anti-MUC16/anti-CD3 bispecific antibody or antigen-binding fragment thereof is administered before, after, or concurrent with the anti-PD-1 antibody or antigen-binding fragment thereof. The term “in combination with” also includes sequential or concomitant administration of anti-PD-1 antibody or antigen-binding fragment thereof and a bispecific anti-MUC16/anti-CD3 antibody or antigen-binding fragment thereof. For example, when administered “before” the bispecific anti-MUC16/anti-CD3 antibody or antigen-binding fragment thereof, the anti-PD-1 antibody or antigen-binding fragment thereof may be administered more than 150 hours, about 150 hours, about 100 hours, about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes or about 10 minutes prior to the administration of the bispecific anti-MUC16/anti-CD3 antibody or antigen-binding fragment thereof. When administered “after” the bispecific anti-MUC16/anti-CD3 antibody or antigen-binding fragment thereof, the anti-PD-1 antibody or antigen-binding fragment thereof may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours, about 72 hours, or more than 72 hours after the administration of the bispecific anti-MUC16/anti-CD3 antibody or antigen-binding fragment thereof. Administration “concurrent” with the bispecific anti-MUC16/anti-CD3 antibody or antigen-binding fragment thereof means that the anti-PD-1 antibody or antigen-binding fragment thereof is administered to the subject in a separate dosage form within less than 5 minutes (before, after, or at the same time) of administration of the bispecific anti-MUC16/anti-CD3 antibody or antigen-binding fragment thereof, or administered to the subject as a single combined dosage formulation comprising both the anti-PD-1 antibody or antigen-binding fragment thereof and the bispecific anti-MUC16/anti-CD3 antibody or antigen-binding fragment thereof.

In certain embodiments, the methods of the present invention comprise administration of a third therapeutic agent wherein the third therapeutic agent is an anti-cancer drug. In certain embodiments, the methods of the invention comprise administering an anti-PD-1 antibody or antigen-binding fragment thereof and an anti-MUC16/anti-CD3 bispecific antibody or antigen-binding fragment thereof in combination with radiation therapy to generate long-term durable anti-tumor responses and/or enhance survival of patients with cancer.

In some embodiments, the methods of the invention comprise administering radiation therapy prior to, concomitantly or after administering an anti-PD-1 antibody or antigen-binding fragment thereof and a bispecific anti-MUC16/anti-CD3 antibody or antigen-binding fragment thereof to a cancer patient. For example, radiation therapy may be administered in one or more doses to tumor lesions after administration of one or more doses of the antibodies (or fragments). In some embodiments, radiation therapy may be administered locally to a tumor lesion to enhance the local immunogenicity of a patient’s tumor (adjuvinating radiation) and/or to kill tumor cells (ablative radiation) after systemic administration of an anti-PD-1 antibody or antigen-binding fragment thereof and/or a bispecific anti-MUC16/anti-CD3 antibody or antigen-binding fragment thereof.

### Pharmaceutical Compositions and Administration

The present invention includes methods which comprise administering a bispecific anti-MUC16/anti-CD3 antibody or antigen-binding fragment thereof alone, or in combination with an anti-PD-1 antibody or antigen-binding fragment thereof to a subject wherein the antibody or antibodies (or fragments) are contained within separate or a combined (single) pharmaceutical composition. The pharmaceutical compositions of the invention may be formulated with suitable carriers, excipients, and other agents that provide suitable transfer, delivery, tolerance, and the like. A multitude of appropriate formulations can be found in the formulary known to all pharmaceutical chemists: Remington’s Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al., 1987, J. Biol. Chem. 262: 4429-4432). Methods of administration include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present invention include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.), and the HUMIRA™ Pen (Abbott Labs, Abbott Park IL), to name only a few.

In certain situations, the pharmaceutical composition can be delivered in a controlled release system. In one embodiment, a pump may be used. In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition’s target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

### Administration Regimens

The present invention includes methods comprising administering to a subject a bispecific anti-MUC16 x CD3 antibody or antigen-binding fragment thereof and/or an anti-PD-1 antibody or antigen-binding fragment thereof at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved.

According to certain embodiments of the present invention, multiple doses of a bispecific anti-MUC16/anti-CD3 antibody or antigen-binding fragment thereof alone, or in combination with an anti-PD-1 antibody or antigen-binding fragment thereof may be administered to a subject over a defined time course. The methods according to this aspect of the invention comprise sequentially administering to a subject one or more doses of a bispecific anti-MUC16/anti-CD3 antibody or antigen-binding fragment thereof alone, or in combination with one or more doses of an anti-PD-1 antibody or antigen-binding fragment thereof. As used herein, “sequentially administering” means that each dose of the antibody or antigen-binding fragment thereof is administered to the subject at a different point in time, e.g., on different days separated by a predetermined interval (e.g., hours, days, weeks or months). The present invention includes methods which comprise sequentially administering to the patient a single initial dose of an antibody or antigen-binding fragment thereof, followed by one or more secondary doses of the antibody or antigen-binding fragment thereof, and optionally followed by one or more tertiary doses of the antibody or antigen-binding fragment thereof.

The terms “initial dose,” “secondary doses,” and “tertiary doses,” refer to the temporal sequence of administration. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the antibody or antigen-binding fragment thereof (anti-PD-1 antibody or bispecific antibody). In certain embodiments, however, the amount contained in the initial, secondary and/or tertiary doses varies from one another (e.g., adjusted up or down as appropriate) during the course of treatment. In certain embodiments, one or more (e.g., 1, 2, 3, 4, or 5) doses are administered at the beginning of the treatment regimen as “loading doses” followed by subsequent doses that are administered on a less frequent basis (e.g., “maintenance doses”). For example, an anti-PD-1 antibody or antigen-binding fragment thereof may be administered to a patient with an ovarian cancer at a loading dose of about 1-3 mg/kg followed by one or more maintenance doses of about 0.1 to about 20 mg/kg of the patient’s body weight.

In one exemplary embodiment of the present invention, each secondary and/or tertiary dose is administered ½ to 14 (e.g., ½, 1, 11/2, 2, 21/2, 3, 31/2, 4, 41/2, 5, 51/2, 6, 61/2, 7, 71/2, 8, 81/2, 9, 91/2, 10, 101/2, 11, 111/2, 12, 121/2, 13, 131/2, 14, 141/2, or more) weeks after the immediately preceding dose. The phrase “the immediately preceding dose,” as used herein, means, in a sequence of multiple administrations, the dose of a bispecific anti-MUC16/anti-CD3 antibody or antigen-binding fragment thereof (and/or anti-PD-1 antibody or antigen-binding fragment thereof) which is administered to a patient prior to the administration of the very next dose in the sequence with no intervening doses.

The methods according to this aspect of the invention may comprise administering to a patient any number of secondary and/or tertiary doses of bispecific anti-MUC16/anti-CD3 antibody or antigen-binding fragment thereof (and/or an anti-PD-1 antibody or antigen-binding fragment thereof). For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

In certain embodiments, one or more doses of a bispecific anti-MUC16/anti-CD3 antibody or antigen-binding fragment thereof (e.g., and an anti-PD-1 antibody or antigen-binding fragment thereof) are administered at the beginning of a treatment regimen as “induction doses” on a more frequent basis (twice a week, once a week or once in 2 weeks) followed by subsequent doses (“consolidation doses” or “maintenance doses”) that are administered on a less frequent basis (e.g., once in 4-12 weeks).

The present invention includes methods comprising sequential administration of a bispecific anti-MUC16/anti-CD3 antibody or antigen-binding fragment thereof alone, or in combination with an anti-PD-1 antibody or antigen-binding fragment thereof to a patient to treat an ovarian cancer (e.g., serous cancer). In some embodiments, the present methods comprise administering one or more doses of a bispecific anti-MUC16/anti-CD3 antibody or antigen-binding fragment thereof, optionally followed by one or more doses of an anti-PD-1 antibody or antigen-binding fragment thereof. In certain embodiments, the present methods comprise administering a single dose of an anti-PD-1 antibody or antigen-binding fragment thereof followed by one or more doses of a bispecific anti-MUC16/anti-CD3 antibody or antigen-binding fragment thereof. For example, one or more doses of about 0.1 mg/kg to about 20 mg/kg of an anti-PD-1 antibody or antigen-binding fragment thereof may be administered followed by one or more doses of about 0.1 mg/kg to about 20 mg/kg of the bispecific antibody or antigen-binding fragment thereof to inhibit tumor growth and/or to prevent tumor recurrence in a subject with an ovarian cancer. In some embodiments, the anti-PD-1 antibody or antigen-binding fragment thereof is administered at one or more doses followed by one or more doses of the bispecific antibody or antigen-binding fragment thereof resulting in increased anti-tumor efficacy (e.g., greater inhibition of tumor growth, increased prevention of tumor recurrence as compared to an untreated subject or a subject administered with either antibody or antigen-binding fragment thereof as monotherapy). Alternative embodiments of the invention pertain to concomitant administration of anti-PD-1 antibody or antigen-binding fragment thereof and the bispecific antibody or antigen-binding fragment thereof which is administered at a separate dosage at a similar or different frequency relative to the anti-PD-1 antibody or antigen-binding fragment thereof. In some embodiments, the bispecific antibody or antigen-binding fragment thereof is administered before, after or concurrently with the anti-PD-1 antibody or antigen-binding fragment thereof. In certain embodiments, the bispecific antibody or antigen-binding fragment thereof is administered as a single dosage formulation with the anti-PD-1 antibody or antigen-binding fragment thereof.

### Dosage

The amount of bispecific anti-MUC16/anti-CD3 antibody or antigen-binding fragment thereof, and optionally anti-PD-1 antibody or antigen-binding fragment thereof, administered to a subject according to the methods of the present invention is, generally, a therapeutically effective amount. As used herein, the phrase “therapeutically effective amount” means an amount of antibody or antigen-binding fragment thereof (anti-PD-1 antibody or bispecific anti-MUC16/anti-CD3 antibody) that results in one or more of: (a) a reduction in the severity or duration of a symptom of a cancer (e.g., ovarian cancer); (b) inhibition of tumor growth, or an increase in tumor necrosis, tumor shrinkage and/or tumor disappearance; (c) delay in tumor growth and development; (d) inhibit or retard or stop tumor metastasis; (e) prevention of recurrence of tumor growth; (f) increase in survival of a subject with cancer (e.g., ovarian cancer); and/or (g) a reduction in the use or need for conventional anti-cancer therapy (e.g., reduced or eliminated use of chemotherapeutic or cytotoxic agents) as compared to an untreated subject or a subject administered with either antibody (or fragment) as monotherapy.

In the case of a bispecific anti-MUC16/anti-CD3 antibody or antigen-binding fragment thereof, a therapeutically effective amount can be from about 0.1 milligrams (mg) to about 1000 mg, e.g., about 0.1 mg, about 0.2 mg, about 0.3 mg, about 0.5 mg, about 1 mg, about 3 mg, about 5 mg, about 10 mg, about 20 mg, about 25 mg, about 30 mg, about 35 mg, about 40 mg, about 45 mg, about 50 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 225 mg, about 250 mg, about 275 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg of the bispecific anti-MUC16/anti-CD3 antibody or antigen-binding fragment thereof. In some cases, the dose is about 1 mg. In some cases, the dose is about 2 mg. In some cases, the dose is about 20 mg. In some cases, the dose is about 25 mg. In some cases, the dose is about 250 mg. In some cases, the dose is about 800 mg. Any of these doses may the initial dose, the intermediate or transitional dose, or the full dose.

In the case of an anti-PD-1 antibody or antigen-binding fragment thereof, a therapeutically effective amount can be from about 0.05 mg to about 600 mg, e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg, of the anti-PD-1 antibody or antigen-binding fragment thereof. In certain embodiments, 350 mg of an anti-PD-1 antibody or antigen-binding fragment thereof is administered.

The amount of bispecific anti-MUC16/anti-CD3 antibody or antigen-binding fragment thereof and optionally anti-PD-1 antibody or antigen-binding fragment thereof contained within the individual doses may be expressed in terms of milligrams of antibody or antigen-binding fragment thereof per kilogram of subject body weight (i.e., mg/kg). In certain embodiments, the bispecific anti-MUC16/anti-CD3 antibody or antigen-binding fragment thereof, and optionally the anti-PD-1 antibody or antigen-binding fragment thereof, used in the methods of the present invention may be administered to a subject at a dose of about 0.0001 to about 100 mg/kg of subject body weight. For example, the bispecific anti-MUC16/anti-CD3 antibody or antigen-binding fragment thereof may be administered at a dose of about 0.1 mg/kg to about 20 mg/kg of a patient’s body weight, and the optional anti-PD-1 antibody or antigen-binding fragment thereof may be administered at dose of about 0.1 mg/kg to about 20 mg/kg of a patient’s body weight.

A summary of the sequences and the corresponding SEQ ID NOs referenced herein is shown in Table 1, below.

TABLE 1

Summary of Sequences

| SEQ ID NO: | Description |
|---|---|
| 1 | Anti-MUC16 Heavy Chain Variable Region |
| 2 | Anti-MUC16 and Anti-CD3 Light Chain Variable Region |
| 3 | Anti-CD3-G Heavy Chain Variable Region |
| 4 | Anti-CD3-G5 Heavy Chain Variable Region |
| 5 | Anti-CD3-G9 Heavy Chain Variable Region |
| 6 | Anti-CD3-G10 Heavy Chain Variable Region |
| 7 | Anti-CD3-G20 Heavy Chain Variable Region |
| 8 | Anti-MUC16 HCDR1 |
| 9 | Anti-MUC16 HCDR2 |
| 10 | Anti-MUC16 HCDR3 |
| 11 | Anti-MUC16 and Anti-CD3 LCDR1 |
| 12 | Anti-MUC16 and Anti-CD3 LCDR2 |
| 13 | Anti-MUC16 and Anti-CD3 LCDR3 |
| 14 | Anti-CD3-G HCDR1 |
| 15 | Anti-CD3-G HCDR2 |
| 16 | Anti-CD3-G HCDR3 |
| 17 | Anti-CD3-G5 HCDR1 |
| 18 | Anti-CD3-G5 HCDR2 |
| 19 | Anti-CD3-G5 HCDR3 |
| 20 | Anti-CD3-G9 HCDR1 |
| 21 | Anti-CD3-G9 HCDR2 |
| 22 | Anti-CD3-G9 HCDR3 |
| 23 | Anti-CD3-G10 HCDR1 |
| 24 | Anti-CD3-G10 HCDR2 |
| 25 | Anti-CD3-G10 HCDR3 |
| 26 | Anti-CD3-G20 HCDR1 |
| 27 | Anti-CD3-G20 HCDR2 |
| 28 | Anti-CD3-G20 HCDR3 |
| 29 | Anti-MUC16 Heavy Chain |
| 30 | Anti-MUC16 and Anti-CD3 Light Chain |
| 31 | Anti-CD3-G Heavy Chain |
| 32 | Anti-CD3-G20 Heavy Chain |
| 33 | Anti-PD-1 Heavy Chain Variable Region |
| 34 | Anti-PD-1 Light Chain Variable Region |
| 35 | Anti-PD-1 HCDR1 |
| 36 | Anti-PD-1 HCDR2 |
| 37 | Anti-PD-1 HCDR3 |
| 38 | Anti-PD-1 LCDR1 |
| 39 | Anti-PD-1 LCDR2 |
| 40 | Anti-PD-1 LCDR3 |
| 41 | Anti-PD-1 Heavy Chain |
| 42 | Anti-PD-1 Light Chain |

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Generation of Bispecific Antibodies That Bind Ovarian Cell-Specific (MUC16) and CD3

The present invention provides bispecific antigen-binding molecules that bind CD3 and MUC16; such bispecific antigen-binding molecules are also referred to herein as “anti-MUC16/anti-CD3 or anti-MUC16xCD3 bispecific molecules.” The anti-MUC16 portion of the anti-MUC16/anti-CD3 bispecific molecule is useful for targeting tumor cells that express MUC16 (also known as CA-125), and the anti-CD3 portion of the bispecific molecule is useful for activating T-cells. The simultaneous binding of MUC16 on a tumor cell and CD3 on a T-cell facilitates directed killing (cell lysis) of the targeted tumor cell by the activated T-cell.

Bispecific antibodies comprising an anti-MUC16-specific binding domain and an anti-CD3-specific binding domain were constructed using standard methodologies, wherein the anti-MUC16 antigen binding domain and the anti-CD3 antigen binding domain each comprise different, distinct HCVRs paired with a common LCVR. In exemplified bispecific antibodies, the molecules were constructed utilizing a heavy chain from an anti-CD3 antibody, a heavy chain from an anti-MUC16 antibody and a common light chain from the anti-MUC16 antibody. In other instances, the bispecific antibodies may be constructed utilizing a heavy chain from an anti-CD3 antibody, a heavy chain from an anti-MUC16 antibody and a light chain from an anti-CD3 antibody or an antibody light chain known to be promiscuous or pair effectively with a variety of heavy chain arms.

Exemplified bispecific antibodies were manufactured having an IgG1 Fc domain (BSMUC16/CD3-001, -002, -003, and -004) or a modified (chimeric) IgG4 Fc domain (BSMUC16/CD3-005) as set forth in U.S. Pat. Application Publication No. US20140243504A1, published on Aug. 28, 2014.

A summary of the component parts of the antigen-binding domains of the various anti-MUC16xCD3 bispecific antibodies constructed is set forth in Table 2.

TABLE 2

Summary of Component Parts of Anti-MUC16xCD3 Bispecific Antibodies

| Bispecific Antibody Identifier | Anti-MUC16 | Anti-CD3 | Common Light Chain Variable Region |
|---|---|---|---|
| | Antigen-Binding Domain | Antigen-Binding Domain | |
| | Heavy Chain Variable Region | Heavy Chain Variable Region | |
| BSMUC16/CD3-001 (also referred to as REGN4018) | (SEQ ID NO:1) | CD3-VH-G (SEQ ID NO:3) | (SEQ ID NO:2) |
| BSMUC16/CD3-002 | (SEQ ID NO:1) | CD3-VH-G5 (SEQ ID NO:4) | (SEQ ID NO:2) |
| BSMUC16/CD3-003 | (SEQ ID NO:1) | CD3-VH-G9 (SEQ ID NO:5) | (SEQ ID NO:2) |
| BSMUC16/CD3-004 | (SEQ ID NO:1) | CD3-VH-G10 (SEQ ID NO:6) | (SEQ ID NO:2) |
| BSMUC16/CD3-005 | (SEQ ID NO:1) | CD3-VH-G20 (SEQ ID NO:7) | (SEQ ID NO:2) |

### Example 2: CA-125 Does Not Interfere With Anti-MUC16xCD3 Antibody Activity In Vitro

The impact of soluble CA-125 (the shed form of MUC16) on the activity of BSMUC16/CD3-001 was assessed using FACS binding and cytotoxicity assays in the presence of high levels of CA-125 purified from ascites of ovarian cancer patients. CA-125 levels are increased in the serum of the majority of ovarian cancer patients and circulating levels could impact any MUC16-targeted therapy by acting as an antigen sink. The levels of CA-125 used in the assay (10,000 U/ml) greatly exceed the median published levels of 656.6 U/mL in ovarian cancer patients. The ability of BSMUC16/CD3-001 to kill MUC16-expressing OVCAR-3 cells in the presence of soluble CA-125 enriched from human ascites (creative Biomart, NY, USA) or a membrane proximal construct expressing the five carboxy-terminal SEA domains and the juxtamembrane region of MUC16 (MUC16Δ) was carried out at an Effector/Target ratio of 4:1 with a fixed concentration of BSMUC16/CD3-001 or CD3-binding control antibody (100 pM), and a serial dilution of either MUC16-1H or MUC16Δ for 72 hours at 37° C. In order to monitor the specific killing of MUC16-bearing target cells, OVCAR-3 cells were labeled with 1 uM of Violet Cell Tracker. After labeling, cells were plated overnight at 37° C. Separately, human PBMCs were plated in supplemented RPMI media at 1×10⁶ cells/mL and incubated overnight at 37° C. in order to enrich for lymphocytes by depleting adherent cells. The next day, target cells were co-incubated with adherent cell-depleted naïve PBMC (Effector/Target cell ratio 4:1) and a serial dilution of either BSMUC16/CD3-001 or the CD3-binding control for 72 hours at 37° C. Cells were removed from cell culture plates using trypsin, and analyzed by FACS. For FACS analysis, cells were stained with a dead/live far red cell tracker (Invitrogen). For the assessment of specificity of killing, cells were gated on Violet cell tracker labeled populations. Percent of live target cells was reported for the calculation of adjusted survival as follows: Adjusted survival=(R1/R2)*100, where R1= % live target cells in the presence of antibody, and R2= % live target cells in the absence of test antibody. T cell activation was assessed by incubating cells with directly conjugated antibodies to CD2, CD69, and CD25, and by reporting the percent of activated (CD69+) T cells or (CD25+) T cells out of total T cells (CD2+).

The binding of BSMUC16/CD3-001 and an antibody known to bind CA-125 (clone 3A5) to CA125 obtained from human ascites fluid was measured by enzyme-linked immunosorbent assay (ELISA). Briefly, soluble CA-125 (creative Biomart, NY, USA) at a concentration of 4000 units/mL in PBS was passively adsorbed to a 96-well microtiter plates overnight at 4° C. The plates were then washed with PBST and blocked with 0.5% BSA in PBS for 1 hour. Biotinylated BSMUC16/CD3-001, the MUC16 parental antibody, a-MUC16 3A5 and non-binding controls (BSMUC16/CD3-001 isotype control and a-MUC16 3A5 isotype control), were added to plate at concentrations of 10, 1, 0.3, or 0.1 nM in 0.5% BSA in PBS for 1 hour, followed by a wash with PBST. Streptavidin conjugated with horseradish peroxidase (SA-HRP) (ThermoFisher Scientific, Waltham, MA, USA) at 1:10000 dilution of 1.0 mg/mL stock solution was added to the wells and incubated for 1 hour to detect plate-bound biotinylated antibodies. The plate was washed and developed with 3-3′, 5-5′-tetramethylbenzidine (BD Biosciences, Franklin Lakes, NJ, USA) substrate according the manufacturer’s instructions. Absorbance at 450 nm was recorded for each well on a Victor Multilabel Plate Reader (Perkin Elmer; Melville, NY). Data were analyzed with GraphPad Prism software.

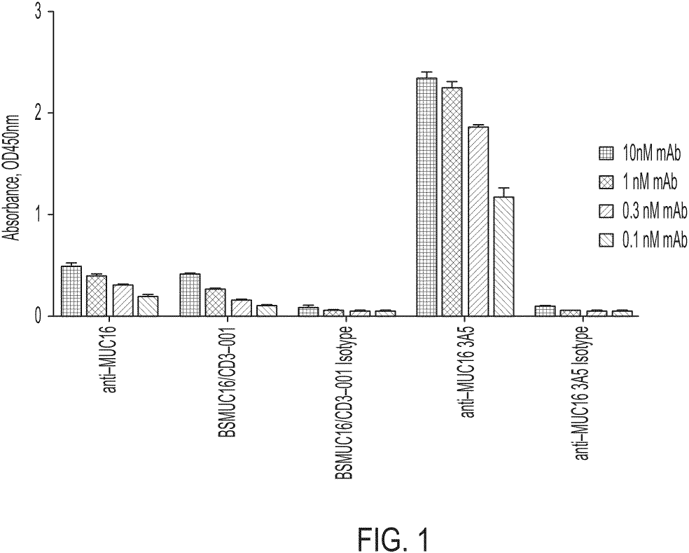

Excess CA-125 had minimal impact on BSMUC16/CD3-0001 binding to OVCAR-3 cells suggesting minimal binding to CA-125 (FIG. 1). In contrast, CA-125 greatly inhibited the ability of a comparator antibody that likely binds to the repeat region of MUC16 (in-house version of antibody clone 3A) (FIG. 1). Further, a soluble MUC16 construct containing the membrane-proximal region up to the 5^{th} SEA domain of MUC16 (MUC16Δ) dramatically inhibited binding of BSMUC16/CD3-001, showing that BSMUC16/CD3-001 binds a membrane proximal region, as discussed in greater detail in WO 2018/067331, which is herein incorporated by reference. In alignment with the binding studies, BSMUC16/CD3-001 could also induce T cell-mediated killing in the presence of CA-125, but not in the presence of a high concentration of MUC16Δ (data not shown). Thus, BSMUC16/CD3-001 can bind to MUC16 and induce T cell redirected killing even in the presence of high concentrations of CA-125.

### Example 3: PD-1 Blockade Enhances Anti-Tumor Activity of Anti-MUC16xCD3 Bispecific Antibodies in Xenogenic and Syngeneic Tumor Models

The in vivo efficacy of an anti-MUC16/anti-CD3 bispecific antibody in combination with PD-1 blockade was evaluated in xenogenic and syngeneic tumor models.

### A. Xenogenic Model - OVCAR-3/Luc

For the xenogenic model, immunodeficient NSG mice were injected intraperitoneally (IP) with OVCAR-3/Luc cells previously passaged in vivo (Day 0) thirteen days after engraftment with human PBMCs. Mice were treated IP with 12.5 ug/mouse BSMUC16/CD3-001, or administered 12.5 ug CD3-binding control alone or in combination with 100 ug REGN2810 on Days 5 and 8. Tumor burden was assessed by BLI on Days 4, 8, 12, 15, 20 and 25 post tumor implantation. As determined by BLI measurements on Day 25, treatment with 12.5 ug of BSMUC16/CD3-001 resulted in significant anti-tumor efficacy as determined by BLI measurements and combination with REGN2810 (anti-PD-1) further enhanced the anti-tumor efficacy. All groups had similar tumor burden as assessed by BLI before dosing started. There was no significant difference in tumor burden between groups.

BSMUC16/CD3-001 significantly reduces tumor burden at 12.5 ug and addition of anti-PD-1 enhances the anti-tumor efficacy over that of BSMUC16/CD3-001 alone. NSG mice engrafted with human T cells were implanted with human OVCAR-3/Luc cells. Mice were treated on Days 5 and 8 with 12.5 ug BSMUC16/CD3-001 administered IV or treated with a CD3-binding control or non-binding control (12.5 ug IV). Data shown in Table 3, below, is tumor burden as assessed by BLI on Day 25 post tumor implantation. Statistical significance was determined using unpaired nonparametric Mann-Whitney t-tests. Treatment with BSMUC16/CD3-001 +/- REGN2810 was compared to the CD3-binding control (* p < 0.05 for BSMUC16/CD3-001, ** p < 0.01 for BSMUC16/CD3-001 and REGN2810) and treatment with BSMUC16/CD3-001 alone was compared to combination with REGN2810 (# p < 0.05).

TABLE 3

Bioluminescence on Day 25 post tumor implantation

| | |
|---|---|
| Antibody (ug) | Avg Radiance [p/s/cm²/sr] 25 days postimplantation (median ± SEM) |
| hIgG4P-PVA CD3-binding Control (12) | 7.71e+06 ± 1.07e+06 |
| BSMUC16/CD3-001 (12) | 7.44e+03 ± 3.11e+03 |
| hIgG4P-PVA CD3-binding Control (12) + REGN2810 (100) | 9.29e+06 ± 1.82e+06 |
| BSMUC16/CD3-001 (12) + REGN2810 (100) | 1.76e+03 ± 9.38e+01 |

### B. Syngeneic Model - ID8-VEGF/huMUC16

To examine efficacy in an immune-competent model, the murine CD3 gene was replaced with human CD3 and a portion of the mouse MUC16 gene was replaced with the human sequence. The replacements resulted in a mouse whose T cells express human CD3 and that expresses a chimeric MUC16 molecule containing a portion of human MUC16 where the BSMUC16/CD3-001 and BSMUC16/CD3-005 bispecific antibodies bind.

For this first syngeneic tumor model, the ID8-VEGF cell line engineered to express the portion of human MUC16 was used. Mice were implanted with the ID8-VEGF/huMUC16 cells IP and treated with 5 mg/kg of BSMUC16/CD3-001 or CD3-binding control with isotype control or in combination with anti-PD-1 (5 mg/kg IV) three days after implantation. Treatment with BSMUC16/CD3-001 extended the median survival compared to the group that received the CD3-binding control but the addition of anti-PD-1 blockade also resulted in survival of 50% of the mice.

BSMUC16/CD3-001 significantly increases median survival time in an ID8-VEGF ascites model and addition of PD-1 (REGN2810) blockade allows survival of several mice. Mice expressing human CD3 in place of mouse CD3 and a chimeric MUC16 molecule were implanted with the murine ovarian tumor line expressing a portion of human MUC16. Mice were administered BSMUC16/CD3-001 (5 mg/kg IV) or administered CD3-binding control (5 mg/kg IV) with isotype control or with anti-PD-1 on day 3 post implantation. Mice were treated on Days 3, 7, 10, 14, 17 post tumor implantation. Data shown is median survival. Mice were sacrificed when they had a with weight-gain of more than 20% due to ascites-induced abdominal distension. Statistical significance was determined using the Mantel-Cox method. Both BSMUC16/CD3-001 and BSMUC16/CD3-001 + anti-PD-1 treatment resulted in an increase in median survival time and the combination of BSMUC16/CD3-001 + anti-PD-1 resulted in 50% survival, demonstrating a synergistic effect between the MUC16xCD3 bispecific antibody and the anti-PD-1 antibody. Results are shown in Table 4, below.

TABLE 4

Median Survival in the ID8-VEGF/huMUC16 model

| Antibody (mg/kg) | Median Survival (Days) |
|---|---|
| CD3-binding control (5) + isotype control (5) | 36 |
| BSMUC16/CD3-001 (5) + isotype control (5) | 46 |
| CD3-binding control (5) + PD-1 (5) | 32 |
| BSMUC16/CD3-001 (5) + PD-1 (5) | 69.5 |

Similar results were observed when BSMUC16/CD3-001 was administered at 1 mg/kg in combination with the anti-PD-1 antibody.

### C. Syngeneic Model - MC38/huMUC16

As discussed above, the mice used in this experiment were engineered so that the murine CD3 gene was replaced with human CD3 and a portion of the mouse MUC16 gene was replaced with the human sequence. The replacements resulted in a mouse whose T cells express human CD3 and that expresses a chimeric MUC16 molecule containing a portion of human MUC16 where the BSMUC16/CD3-001 and BSMUC16/CD3-005 bispecific antibody binds.

For this second syngeneic tumor model, the MC38 line engineered to express the portion of human MUC16 was used. Mice were implanted with MC38/huMUC16 cells SC and treated with BSMUC16/CD3-005 or CD3-binding control with isotype control (1 mg/kg IV) or in combination with anti-PD-1 (5 mg/kg IV) on Day 7 post tumor implantation. The anti-PD-1 antibody used in this experiment was a commercially available murine antibody (clone RMP1-14, BioXCell). The combination of BSMUC16/CD3-005 and anti-PD-1 showed a synergistic anti-tumor effect.

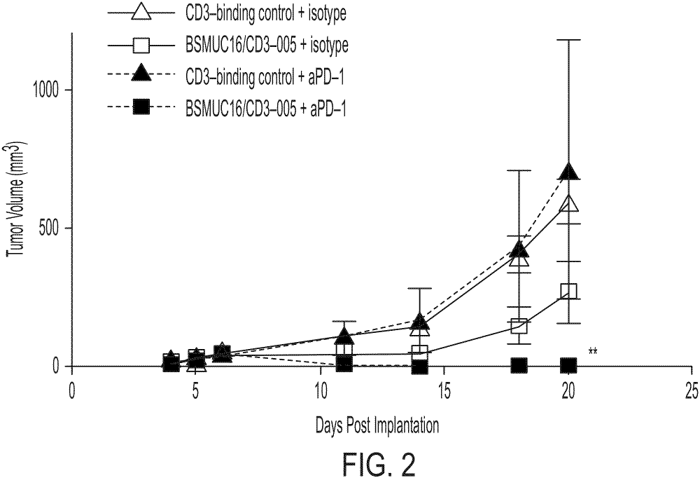

The combination of BSMUC16/CD3-005 and anti-PD-1 blockade resulted in better anti-tumor efficacy than BSMUC16/CD3-005 alone in a MC38 SC model. Mice expressing human CD3 in place of mouse CD3 and a chimeric MUC16 molecule were implanted with the murine tumor line MC38 expressing a portion of human MUC16. Mice were administered BSMUC16/CD3-005 or administered CD3-binding control (1 mg/kg IV) with isotype control or with anti-PD-1 antibody (5 mg/kg IV) on day 7 post implantation. Mice were treated on Days 7, 11 and 14 post tumor implantation. The results are illustrated in FIG. 2. Statistical significance was determined using two-way ANOVA with Tukey’s multiple comparison test. BSMUC16/CD3-005 plus anti-PD-1 significantly, and synergistically, inhibited tumor growth over the CD3-binding control.

### Example 4: Immuno-PET Imaging in Engineered Mice Showed Localization of the Anti-MUC16xCD3 Bispecific Antibody to T Cell-Rich Organs

The in vivo localization of BSMUC16/CD3-001 and BSMUC16/CD3-005 and the expression of MUC16 protein were assessed in wild type and genetically humanized mice using PET imaging. The biodistribution of the ⁸⁹Zr-labelled anti-MUC16 antibody (bivalent anti-MUC16 antibody generated using the same anti-MUC16 heavy and light chain as the bispecifics, herein referred to as “parental”) was similar in both wild type and humanized mice, suggesting low expression/availability of the humanized MUC16 protein to the antibody. In contrast, when mice were administered therapeutically relevant doses of a ⁸⁹Zr-labelled BSMUC16/CD3-001 bispecific antibody, distribution to the spleen and lymph nodes was evident due to recognition of CD3 positive T cells in these lymphoid organs (data not shown). Ex vivo biodistribution analyses in individual tissues confirmed localization to lymph nodes and spleen (data not shown). Uptake of ⁸⁹Zr-labelled BSMUC16/CD3-005 bispecific antibody in lymphoid tissues was greatly reduced relative to BSMUC16/CD3-001 due to its lower affinity for CD3. To assess whether BSMUC16/CD3-001 and BSMUC16/CD3-005 can accumulate in MUC16-expressing tumors, ⁸⁹Zr-labelled BSMUC16/CD3-001 and ⁸⁹Zr-labelled BSMUC16/CD3-005 were administered to mice bearing ID8-VEGF-huMUC16Δ tumors. Tumor uptake between the bispecific antibodies was not significantly different despite the higher lymphoid uptake of BSMUC16/CD3-001 (data not shown).

Preparation of immunoconjugate and small animal PET: BSMUC16/CD3-001 and control antibody were conjugated with DFO to glutamine residues at position 295 via transamidation by microbial transglutaminase following deglycosylation of the antibodies with PNGase F. DFO conjugated antibodies were then chelated with Zirconium-89 (⁸⁹Zr). Mice received antibody at a final dose of 0.5 mg/kg via tail vein injection. PET imaging was then performed to assess in vivo localization of the radioimmunoconjugate at day 6 post dosing, prior to ex vivo biodistribution studies. For experiments in tumor-bearing mice, mice were implanted subcutaneously with 10×10⁶ ID8-VEGF-huMUC16Δ tumor cells. Tumor bearing mice were dosed with ⁸⁹Zr radiolabeled antibodies 20 day post implantation when tumors averaged 150 mm³.

A pre-calibrated Sofie Biosciences G8 PET/CT instrument (Sofie Biosciences (Culver city, CA) and Perkin Elmer) was used to acquire PET and CT images. The energy window ranged from 150 to 650 keV with a reconstructed resolution of 1.4 mm at the center of the field of view. On day 6 post dosing, mice underwent induction anesthesia using isoflurane and were kept under continuous flow of isoflurane during a 10-minute static PET acquisition. CT images were acquired following PET acquisition. The PET image was subsequently reconstructed using pre-configured settings. Decay-corrected PET data and CT data were processed using VivoQuant software (inviCRO Imaging Services) into false-colored co-registered PET-CT maximum intensity projections on a color scale calibrated to indicate a signal range of 0 to 30% of injected dose per volume, expressed as %ID/g. For ex vivo biodistribution analysis, mice were euthanized following imaging on day 6 post dosing. Blood was collected via cardiac puncture into counting tubes. Normal tissues (inguinal and axillary lymph nodes, thymus, spleen, heart, lungs, stomach, small intestine, liver, kidneys, bone and ovary) were then excised and placed into counting tubes. Tumors were similarly collected into counting tubes. All tubes had been pre-weighed and were subsequently re-weighed to determine the weight of the blood and tissues. The _{Y}-emission radioactivity for all samples were then counted on an automatic gamma counter (Wizard 2470, Perkin Elmer) and results reported in in counts per minute (cpm). The %lD for each sample was the determined using samples counts relative to dose-standards counts prepared from the original injected material. Subsequently, the individual %ID/g values were derived by dividing the %ID value by the respective weight of the appropriate blood, tissues or tumor sample.

⁸⁹Zr-labeled BSMUC16/CD3-001 and ⁸⁹Zr-labeled BSMUC16/CD3-005 demonstrated specific localization to MUC16+ tumors and CD3+ lymphoid tissues, with lymphoid distribution correlating to relative CD3 affinity. Both MUC16xCD3 bispecifics demonstrated equivalent tumor localization in the presence of CD3+ tissues.

### Example 5: Toxicology Studies in Cynomolgus Monkeys Showed No Overt Toxicity For the Anti-MUC16xCD3 Bispecific Antibody

BSMUC16/CD3-001 cross-reacts with monkey MUC16 and CD3. To determine the safety and tolerability, and characterize the pharmacokinetics of the bispecific antibody, a multidose toxicity study was conducted in cynomolgus monkeys. Six monkeys/sex/group received weekly administration of BSMUC16/CD3-001 for a total of five doses at 0.01, 0.1 or 1 mg/kg. At the completion of the dosing period, 3 animals/sex/group were euthanized and tissues examined for microscopic finding, while the remaining three animals/sex/group underwent 12 weeks of treatment-free recovery to assess the reversibility or persistence of any BSMUC16/CD3-001-related effects. BSMUC16/CD3-001 was well tolerated, and all animals survived to the time of scheduled necropsy. Toxicokinetic analysis demonstrated doseproportional exposures and linear kinetics across the dose groups, with no gender differences observed (data not shown). Continuous exposure to BSMUC16/CD3-001 was observed throughout the dosing phase, and BSMUC16/CD3-001 exposure was maintained until the end of the recovery phase in all (n=6) and 50% of animals in the 0.1 and 1.0 mg/kg groups, respectively. BSMUC16/CD3-001 was not detected in the serum in any animal in the 0.01 mg/kg group after recovery week 8. The elimination half-life of BSMUC16/CD3-001 was approximately 10 days.

There were no BSMUC16/CD3-001-related clinical observations, nor any changes in urinalysis parameters, peripheral blood immunophenotyping, food consumption, or body weight during the dosing or recovery periods. Importantly, BSMUC16/CD3-001 administration did not result in any changes in respiratory, neurologic, or cardiovascular safety pharmacology evaluations, including no changes in ECG parameters. No BSMUC16/CD3-001-related changes in organ weight were found, nor were any macroscopic changes noted at either terminal or recovery necropsy. Dose-related, reversible elevations of circulating inflammatory markers (C-reactive protein (CRP) and IL-6) were observed within 1 day after the initial dose of either 1.0 or 0.1 mg/kg, but these elevations were not apparent after subsequent doses (data not shown). In accordance with the minimal increase of serum cytokines, T cell redistribution was not detected after BSMUC16/CD3-001 administration (data not shown), in contrast to what has been described for several CD3 bispecific molecules against hematological tumors.

The cynomolgus monkey study was conducted in accordance to guidelines of the IACUC. Cynomolgus monkeys (6 animals/sex/group) were administered control article (diluted placebo) or BSMUC16/CD3-001 (0.01, 0.1, or 1 mg/kg) once weekly via a 30-minute IV infusion. The control article was 10 mM histidine with 10% sucrose and 0.05% polysorbate 20, pH 6, diluted with 0.9% sodium chloride for injection, USP (sterile saline). Blood samples or tissues were collected at various time points for clinical pathology and histopathology. BSMUC16/CD3-001 concentration was determined by ELISA and toxicokinetic analysis was performed using WinNonLin software. CRP was analyzed on a Roche Modular P 800 system. Cytokines were measured by MSD (Meso Scale Diagnostics, Rockville, MD). T cells were quantitated using flow cytometry. Briefly, blood was collected in potassium EDTA tubes, lysed, stained for CD3, CD4 and CD8 (BD Biosciences) and relative values for each phenotype are determined using a FACS Canto II. These values are then multiplied by the absolute lymphocyte values (via hematology analysis) to enumerate absolute cell counts for each phenotype.

Immunohistochemical staining for MUC16 was present in expected tissues: pancreas (mesothelium, ductal epithelium), heart and ovary (data not shown) as well as salivary gland (goblet cells), liver (mesothelium, bile duct), lung (mesothelium, bronchiolar/bronchial epithelium), small intestine (mesothelium), testis (mesothelium, rete testis/efferent duct) and tonsil (epithelium, mucous glands) (not shown). BSMUC16/CD3-001-related microscopic changes, evaluated by hematoxylin and eosin (H&E) histologic staining, included inflammation (infiltration of white blood cells) and increased mesothelial cell size and cellularity leading to non-adverse thickening of the serosal lining and/or submesothelial connective tissue of multiple thoracic and peritoneal organs. These changes were generally focal or multi-focal in nature and were minimal to slight in severity and were considered to be on-target for BSMUC16/CD3-001, resulting from engagement of MUC16 expressed on serosal epithelial (mesothelial) cells and activation of T cells. Importantly, the serosal changes were reversed or trended towards reversal at the end of the recovery period (data not shown).

Toxicology studies in cynomolgus monkeys showed minimal and transient increases in serum cytokines and C-reactive protein following BSMUC16/CD3-001 administration, with no overt toxicity.

### Example 6: Assessment of Serum Cytokine Induction in Tumor-Bearing Mice

Because cytokine release syndrome (CRS) is a frequent serious side effect of CD3 bispecific and CAR T cell therapies, a study to monitor serum cytokines in relevant models following treatment with BSMUC16/CD3-001 was conducted. In genetically humanized MUC16/CD3 mice without tumors, no serum cytokine response was evident upon BSMUC16/CD3-001 administration.

To assess in vivo T cell activation by BSMUC16/CD3-001, serum cytokine levels from tumor-bearing mice were measured. Serum samples were collected 4 hours after the first antibody dose in the 0.5 mg/kg BSMUC16/CD3-001, CD3-binding control, and non-binding control groups. Treatment with BSMUC16/CD3-001 activated T cells as determined by induction of IFN_{Y}, TNFα, IL-2, IL-6, IL-8, and IL-10, compared to the non-binding control and the CD3-binding control (data not shown). BSMUC16/CD3-001-induced cytokine response required the presence of both T cells as well as OVCAR-3/Luc cells, as mice bearing only OVCAR3/Luc cells did not have detectable human IFN_{Y} in the serum, and mice without tumor cells to provide MUC16 for cross-linking did not show an increase in serum IFN_{Y} in response to BSMUC16/CD3-001 (data not shown).

Measurement of serum cytokine levels: T cell activation in response to treatment with BSMUC16/CD3-001 was assessed by measuring the serum concentrations of interferon _{Y} (IFN_{Y}), tumor necrosis factor α (TNFα), interleukin-2 (IL-2), IL-4, IL-6, IL-8, IL-10, IL-12p70, IL-13, and IL-1B four hours after the first 0.5 mg/kg dose. Cytokine levels were analyzed using V-plex Human Prolnflammatory-10 Plex kit following the manufacturer’s instructions (Meso Scale Diagnostics, Rockville, MA). Cytokines were measured in two separate studies with 4-6 mice per group.

### Example 7: MUC16 Expression in Humanized Mice and Effect of Anti-MUC16xCD3 Bispecific Antibodies on MUC16-Positive Tissues

To investigate the antitumor efficacy of BSMUC16/CD3-001 in a mouse with a fully intact immune system, mice were genetically engineered to express human CD3 on T cells and a region of MUC16 covering the antibody binding region, both in the endogenous murine loci (knock-in mice). To validate these mice, MUC16 expression was examined by both RT-PCR and IHC. RNA expression was detected in the trachea as well as low levels in the lung, heart, ovary, pancreas and bladder (data not shown), similar to published data on murine MUC16 expression. To assess MUC16 protein expression, IHC was performed on selected tissues using an anti-human MUC16 antibody that recognizes a membrane-proximal region of MUC16. MUC16 protein expression was confirmed in the surface epithelium of the ovary and stomach in these mice. MUC16 was also observed in the tracheal lining/epithelium as well as the submucosal glands, as has been described in humans (data not shown).

Histology on mouse tissues: Tissues from humanized or WT mice were harvested and stained with an anti-MUC16 antibody binding the membrane proximal domain of MUC16 by IHC using the Ventana Discovery XT (Ventana; Tucson, AZ). 5 µm Paraffin sections were cut onto Superfrost PLUS slides and baked for an hour at 60° C. The immunohistochemical staining was performed on the Discovery XT Automated IHC staining system using the Ventana DAB Map detection kit. Deparaffinization was performed using EZ Prep solution at 75° C. for 8 minutes. Mild antigen retrieval was performed (95° C., 8 minutes followed by 100° C., 24 minutes) using Tris-EDTA buffer pH 9 (CC1) from Ventana. This was followed by multiple blocking steps. Tissue sections were incubated with the anti-MUC16 antibody (2 µg/ml) for 8 hours at RT. An isotype control antibody recognizing an irrelevant non-binding antibody was used as the negative control. Primary antibody and negative control were applied manually. Biotinylated Goat Anti-Human IgG (Jackson ImmunoResearch) was used as the secondary antibody (1 µg/ml) and samples were incubated for an hour at RT. The chromogenic signal was developed using the Ventana DAB MAP Kit. Slides were manually counterstained with Hematoxylin (2 minutes), dehydrated and coverslipped. Images were acquired on the Aperio AT 2 slide scanner (Leica Biosystems; Buffalo Grove, IL) and analyzed using Indica HALO software (Indica Labs; Corrales, NM). H&E staining were performed by Histoserv, Inc (Germantown, MD, USA).

The T cells in these mice are polyclonal, as assessed by T cell receptor (TCR) Vß usage, express human CD3, and are present in similar numbers to wildtype mice (data not shown). To determine whether BSMUC16/CD3-001 induced any T cell activation or effects on normal tissues in these animals, non-tumor-bearing mice were injected with a high dose of BSMUC16/CD3-001 (10 mg/kg) and T cell numbers in blood, serum cytokines, and histopathology were then examined. Although T cells can be activated by an anti-human CD3 antibody (OKT3) as measured by T cell margination from the blood and increased levels of serum cytokines (data not shown), BSMUC16/CD3-001 did not induce any such effects, suggesting limited accessibility of the MUC16 target (data not shown). To determine whether BSMUC16/CD3-001 induced any microscopic changes in MUC16-expressing tissues, MUC16 and CD3 humanized mice received two doses of BSMUC16/CD3-001 at 10 mg/kg on Day 0 and Day 3. On day 5, several MUC16-expressing tissues (trachea, stomach and ovary) were examined, and no cellular infiltration or necrosis was seen in these tissues following BSMUC16/CD3-001 administration (data not shown).

Histopathology examination revealed no inflammation or infiltration into MUC16-expressing tissues in mice after BSMUC16/CD3-001 administration at the time examined.

The results of this study, as well as the cynomolgus monkey study discussed in Example 5, demonstrate the safety profile of BSMUC16/CD3-001. BSMUC16/CD3-001 induced only minimal serum cytokines and, while there was focal induction of inflammation and thickening of the serosal lining in MUC16-expressing suggesting on-target activity, these effects were resolving by the end of the recovery period and consistent with inflammation and increased cellularity indicative of repair. The observed serosal changes were not correlated with any clinical observations, clinical pathology (except inflammatory response), or microscopic changes to the underlying parenchyma. Thus, studies in both genetically humanized mice and cynomolgus monkey show BSMUC16/CD3-001 was well-tolerated.

### Example 8: Monitoring PD-1 Expression in a FACS-Based Cytotoxicity Assay Using Naïve Human Effector Cells

In order to monitor the specific killing of Muc16-bearing target cells by flow cytometry, the ovarian cell line OVCAR-3 was labeled with 1 uM of Violet Cell Tracker. After labeling, cells were plated overnight at 37° C. Separately, human PBMCs were plated in supplemented RPMI media at 1×10⁶ cells/mL and incubated overnight at 37° C. in order to enrich for lymphocytes by depleting adherent macrophages, dendritic cells, and some monocytes. The next day, target cells were co-incubated with adherent cell-depleted naïve PBMC (Effector/Target cell 4:1) and a serial dilution of either BSMUC16/CD3-001 or the CD3-binding control for 72 hours at 37° C. Cells were removed from cell culture plates using trypsin, and analyzed by FACS. For FACS analysis, cells were stained with a dead/live far red cell tracker (Invitrogen). For the assessment of specificity of killing, cells were gated on Violet cell tracker labeled populations.

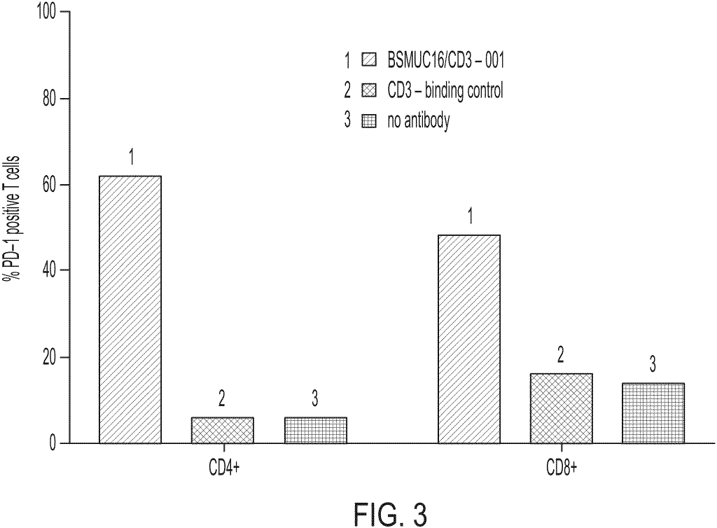

PD-1 expression was assessed by incubating cells with directly conjugated antibodies to CD2, CD4, CD8, and PD-1 by reporting the percent of PD-1/D4 positive T cells or PD-1/D8 positive T cells out of total T cells (CD2+). Incubation with BSMUC16/CD3-001 increased the percentage of PD-1+ T cells by more than 10-fold (CD4+ T cells) or more than 3-fold (CD8+ T cells) compared to controls. Results are shown in FIG. 3.

### Example 9: Methods of Treating Ovarian Cancer With Anti-MUC16 X Anti-CD3 Bispecific Antibodies Alone or in Combination With Anti-PD-1 Antibody

A phase ½ study of the safety, tolerability, preliminary anti-tumor activity, and pharmacokinetics (PK) of REGN4018 (anti-MUC16 x anti-CD3 bispecific antibody) in patients with recurrent ovarian cancer who have exhausted all therapeutic options, including platinum-containing therapy, is being conducted, and is showing a meaningful clinical benefit.

Patients with difficult-to-treat, advanced, platinum-experienced and/or intolerant epithelial ovarian cancer (except carcinosarcoma), primary peritoneal cancer, or fallopian tube cancer are being studied as part of the REGN4018 study.

In ovarian cancer clinical trials, treatment assessment is based on tumor response. The tumor response assessment is based on the levels of CA-125, a tumor marker in patients with and without measurable disease. Additionally, biomarker analysis includes peripheral T-cell phenotyping as REGN4018 treatment is expected to transiently reduce the population of peripheral CD3 T cells. Further analysis will include biomarkers such as tumor expression of proteins such as MUC16 and PD-L1 and may include ctDNA, tumor (RNA and somatic DNA sequencing) genetic analyses for variations that impact the clinical course of underlying disease or modulate treatment side effects.

Objectives: Both primary and secondary endpoints will be explored.

The primary objectives of the study are:
(1) In the dose escalation phase of the study: To assess the safety and pharmacokinetics (PK) in order to determine a maximally tolerated dose (MTD) or recommended phase 2 dose (RP2D) of REGN4018 as monotherapy and in combination with cemiplimab in patients with relapsed ovarian cancer who have exhausted all therapeutic options that are expected to provide meaningful clinical benefit. The determination of the RP2D will be based on the review of nonclinical and all clinical data, including that pertaining to safety, pharmacokinetics (PK), and PK/PD (pharmacokinetic/pharmacodynamic) relationships.
(2) In the dose expansion phase of the study: To assess the preliminary efficacy of REGN4018 as monotherapy and in combination with cemiplimab, (separately by cohort) as determined by the objective response rate (ORR) by Response Evaluation Criteria in Solid Tumors (RECIST 1.1).

The secondary objectives of the study are (in both the dose escalation and dose expansion phases):
(1) To assess the preliminary efficacy of REGN4018 as monotherapy and in combination with cemiplimab, as measured by ORR, best overall response (BOR), duration of response (DOR), progression-free survival (PFS), disease control rate, and CR rate;
(2) To assess efficacy of REGN4018 as monotherapy and in combination with cemiplimab as measured by CA-125 level;
(3) To characterize the immunogenicity of REGN4018 and cemiplimab;
(4) In the dose escalation phase only: to assess the preliminary efficacy of REGN4018 as monotherapy and in combination with cemiplimab, as measured by ORR; and
(5) In the dose expansion phase only: to evaluate the safety, PK, and tolerability of REGN4018 as monotherapy and in combination with cemiplimab.

The exploratory objectives of the study are:
(1) To assess preliminary efficacy of REGN4018 as monotherapy and in combination with cemiplimab (separately by cohort) as measured by ORR based on combined assessment of RECIST and CA-125 using the Gynecologic Cancer Intergroup (GCIG) Criteria;
(2) To evaluate biomarkers that may correlate with mechanism of action, increased understanding of disease/target, observed toxicity, and potential anti-tumor activity including, but not limited, to:
   Circulating proteins
   Circulating immune cells
   Gene expression changes in peripheral blood and tumor
   Tumor expression levels of proteins such as MUC16 and programmed cell death ligand 1 (PD-L1)
(3) Assessments of both tumor mutation burden and circulating tumor DNA;
(4) Where possible, to evaluate the relationship between exposure and efficacy and safety endpoints; and
(5) Overall survival (OS).

Study Design: A modified 3+3 dose escalation design (“4+3”) with accelerated escalation (n=1 patient per cohort) at the lowest 2 dose levels (DLs) of REGN4018 monotherapy will be utilized. The dose escalation of REGN4018 monotherapy will proceed until a maximum tolerated dose (MTD) is attained, or a dose is selected for expansion based on tolerability and sufficient evidence of activity (RP2D).

A series of DLs of REGN4018 will be investigated as monotherapy. Dose escalation will begin at DL1 with a week 1 dose of 0.1 mg intravenous (IV) and a week 2 dose of 0.3 mg IV (if week 1 is tolerated). DL2 will use a week 1 dose of 0.3 mg IV and a week 2 dose of 1 mg IV (if week 1 is tolerated). Dose escalations will then proceed through DL3, DL4, DL4a, and subsequent DLs unless a dose limiting toxicity (DLT), severe infusion-related reaction (IRR), or CRS is identified.

Thus, each REGN4018 DL will consist of an initial dose, and (provided the initial dose was tolerated) a higher full dose. To minimize the risk of CRS, the initial dose has been set at a maximum of 1 mg for all patients assigned to DLs from DL4/DL4a and above and beginning at DL5a a mandatory transitional dose of 20 mg will be instituted between the initial dose and the full dose. In the interest of patient safety, intermediate dose levels may be added if necessary.

The combination therapy of REGN4018 and cemiplimab was initiated at DLC2 which was considered to be a tolerable and pharmacologically active dose of REGN4018 monotherapy. For the REGN4018 and cemiplimab combination therapy, patients enrolled were dose escalated according to the 4+3 dose escalation design as with monotherapy patients. In order to identify the presumptive RP2D/MTD more effectively and minimize the chance of exposing patients to subtherapeutic and/or overly toxic doses, continued escalation of dose levels (DLs) will follow the BOIN design.

Combination therapy will not escalate above the monotherapy MTD. Study conduct for combination therapy cohorts will occur in a similar fashion as in monotherapy cohorts, and will include a monotherapy lead-in cycle in which REGN4018 is gradually introduced.

Following DLC4, the transitional dose and subsequent dose escalation path selected will follow in accordance with monotherapy transitional dose. For example, if 20 mg transitional dose is used in monotherapy cohorts, the 20 mg transitional dose will be used in combination therapy cohorts. Accordingly, dose escalation will continue to DLC4a and (provided safety data from DLC4a is deemed appropriate) then to DLC5a-DLC9a.

A single full dose of REGN4018 must be tolerated without CRS in order for the patient to begin cycle 2 with cemiplimab combination therapy. Combination cycle 1 will last 4-5 weeks, until patients receive at least one full dose of REGN4018 without the development of CRS.

A monotherapy expansion cohort will be enrolled after identification of the REGN4018 MTD and/or RP2D, and a second combination therapy expansion cohort will be enrolled after identification of the MTD and/or RP2D of REGN4018 in combination with cemiplimab. Dose levels of REGN4018 may differ between monotherapy and combination therapy expansion cohorts. In addition, dosing will be evaluated through a 3-arm, randomized phase 2 cohort evaluating three doses: REGN4018 250 mg IV Q3W; REGN4018 800 mg IV Q3W as monotherapy; and REGN4018 250 mg (or highest tolerable dose in combination with cemiplimab if 250 mg QW is not tolerable) IV Q3W combined with cemiplimab 350 mg IV Q3W.

The DLT observation period for determining safety for dose escalation is defined differently for the monotherapy and combination therapy cohorts. The intent of the DLT observation period for monotherapy cohorts is to monitor the safety and tolerability of a minimum of 2 full doses of REGN4018 during monotherapy dose escalation. For DL1 through DL4, because no transitional dose is used, the DLT window is 28 days. For DL5a/DLC5a and all higher DLs, the DLT observation period is 28 to 35 days (depending on when the second full dose is administered) starting with cycle 1, day 1.

The combination therapy DLT observation period is defined as 21 days of combination therapy starting with cycle 2, day 1, with the intent to monitor the safety and tolerability of the first 3 weeks of REGN4018 and cemiplimab combination therapy. The DLT observation period for determining safety for dose escalation in combination therapy need not evaluate REGN4018 monotherapy, since at each combination therapy DL, the REGN4018 dose would have previously been deemed tolerable during REGN4018 monotherapy dose escalation.

In the phase 1 portion of this study, an additional cohort will explore subcutaneous (SC) administration of the initial and transitional doses of REGN4018 only to assess whether SC route of administration is associated with reduced acute toxicities such as CRS. The full dose of REGN4018 for this cohort (as monotherapy or in combination with cemiplimab) will be selected for this cohort based on a review of the safety and efficacy data demonstrated to date.

Study Duration: The screening period is up to 28 days for all patients. In case of REGN4018 monotherapy, each cycle will be 6 weeks (42 days) long. In case of combination therapy with REGN4018 and cemiplimab, first cycle will be 28 or 35 days long, based on when patients tolerate a full dose of REGN4018 without CRS. Subsequent cycles of the combination therapy will be 6 weeks (42 days) long. The treatment will be continued until either disease progression, intolerable adverse events, withdrawal of consent, or other treatment withdrawal criterion is met. Post-treatment follow up will be either approximately 90 days (core follow-up) or 168 days (surveillance follow-up), based on the reason for treatment cessation.

Study Population: Up to 554 patients (292 in the dose escalation phase, 12 in the exploratory SC cohort, and 250 in the expansion cohorts) are expected to be enrolled. The actual number of patients enrolled will depend on observed DLTs during the monotherapy and combination therapy dose escalation cohorts, the number of additional patients added, whether the second stage (of the Simon 2-stage design) is enrolled for each expansion cohort, and whether trigger events occur during dose expansion. This study will enroll patients with platinum-experienced and/or intolerant ovarian, fallopian tube, or primary peritoneal cancer with elevated (≥ 2x upper limit of normal) serum CA-125 levels.

Inclusion Criteria - A patient must meet the following criteria to be eligible for inclusion in the study:
1. Women age 18 years or greater
2. Patients with histologically or cytologically confirmed diagnosis of advanced epithelial ovarian cancer (except carcinosarcoma), primary peritoneal, or fallopian tube cancer who have all of the following:
   a. Serum CA-125 level ≥2x upper limit of normal (ULN) (in screening),
   b. Received at least 1 line of platinum-containing therapy or must be platinum-intolerant,
   c. Documented relapse or progression on or after the most recent line of therapy, and
   d. No standard therapy options likely to convey clinical benefit
3. Willing and able to comply with clinic visits and study-related procedures Notes:
   a. In escalation cohorts, patients must provide either a newly obtained biopsy (newly obtained biopsies at screening are required unless medically inappropriate and discussed with medical monitor) or archived tumor tissue.
   b. In expansion cohorts, patients must provide a fresh tumor biopsy in screening.
4. Expansion cohorts only: Must have progression on prior therapy documented radiographically and must have at least 1 measurable lesion (not previously irradiated) that can be accurately measured by Response Evaluation Criteria in Solid Tumors (RECIST).
5. Eastern Cooperative Oncology Group (ECOG) performance status of 0 or 1
6. Adequate organ and bone marrow function as follows:
   a. Hemoglobin ≥9.0 g/dL
   b. Absolute neutrophil count ≥1.5 × 10⁹/L
   c. Platelet count ≥75 × 10⁹/L
   d. Serum creatinine ≤1.5x ULN or estimated glomerular filtration rate >50 mL/min/1.73 m² (dose escalation cohorts) or estimated glomerular filtration rate >30 mL/min/1.73 m² (dose expansion cohorts)
   e. Total bilirubin ≤1.5x ULN
   f. Aspartate aminotransferase (AST) and alanine aminotransferase (ALT) ≤3x ULN or ≤5x ULN, if liver metastases
   g. Alkaline phosphatase ≤2.5x ULN (or ≤5.0x ULN, if liver or bone metastases)
7. Life expectancy of at least 3 months.
8. Provide informed consent signed by study patient or legally acceptable representative.
9. Randomized phase 2 expansion cohorts only:
   a. Platinum resistant ovarian cancer meeting 1 of the following criteria:
      Patients who have only had 1 line of platinum-based therapy must have received at least 4 cycles of platinum, must have had a response (complete response/remission [CR] or partial response/remission [PR]) and then progressed between 0 months to 6 months after the date of the last dose of platinum.
      Patients who have received 2 or 3 lines of platinum therapy must have progressed on or within 6 months after the date of the last dose of platinum.
   b. Prior treatment with PARP inhibitor for patients with BRCA mutations or known to be deficient in homologous recombination repair (HRD), or ineligible to receive PARP inhibitor
   c. Prior treatment with bevacizumab or ineligible to receive bevacizumab

Exclusion Criteria - A patient who meets any of the following criteria will be excluded from the study:
1. Currently receiving treatment in another therapeutic study or has participated in a study of an investigational agent and received treatment, or used an investigational device within 4 weeks of first dose of study therapy, or received treatment with an approved systemic therapy within 3 weeks of first dose of study therapy, or has received any previous systemic therapy within 5 half-lives of first dose of study therapy (whichever is longer). Patients who have received or are enrolled in a study involving treatment with a minimal dose of an investigational immunoPET reagent are not excluded. Patients previously treated with bevacizumab are permitted after discussion with the sponsor, if no history of bowel perforation or wound complications on bevacizumab AND last dose >30 days from the first dose of REGN4018, and other non-investigational nonimmunomodulatory antibodies with half-lives longer than 7 days are permitted after a discussion with the sponsor if at least 3 half-lives have elapsed since last treatment.
2. Prior anti-cancer immunotherapy as described below:
   a. Prior treatment with anti-PD-1/D-L1 therapy given within 5 half-lives of first dose Note: In combination therapy cohorts and in the randomized phase 2 cohort, patients who previously discontinued anti-PD-1/D-L1 therapy due to toxicity will also be excluded.
   b. Prior CAR-T cell therapy within 30 days of first dose of study drug
3. Prior treatment with a MUC16-targeted therapy.
4. Expansion cohort only: More than 4 prior lines of cytotoxic chemotherapy. Note: In dose-escalation cohorts there is no maximum limit on prior lines of therapy to be eligible.
5. Corticosteroid therapy (>10 mg prednisone/day or equivalent) within 1 week prior to the first dose of study drug. Patients who require a brief course of steroids (up to 2 days in the week before enrollment) are not excluded.
6. Treatment-related immune-mediated AEs from immune-modulatory agents (including but not limited to anti-PD-1/D-L1 or anti-CTLA-4 monoclonal antibodies or PI3Kdelta inhibitors) that have not resolved to baseline at least 30 days prior to initiation of treatment with study therapy. Note: Endocrine immune-mediated AEs controlled with hormonal or other non-immunosuppressive therapies (without resolution) or grade 1 irAEs affecting any organ system with resolution prior to enrollment are allowed.
7. Expansion cohorts only: another malignancy that is progressing or requires active treatment with the exception of non-melanoma skin cancer that has undergone potentially curative therapy or in situ cervical carcinoma, or any other tumor that has been deemed to be effectively treated with definitive local control (with or without continued adjuvant hormonal therapy) for at least 2 years prior to enrollment.
8. Untreated or active primary brain tumor, CNS metastases, or spinal cord compression. Patients with previously treated central nervous system metastases or spinal cord compression may participate provided they are stable (ie, without evidence of progression by imaging for at least 4 weeks prior to the first dose of study treatment, and any neurologic symptoms have returned to baseline), and there is no evidence of new or enlarging central nervous system metastases, and the patient does not require any systemic corticosteroids for management of central nervous system metastases or spinal cord compression within 2 weeks prior to the first dose of study therapy.
9. Encephalitis, meningitis, or uncontrolled seizures in the year prior to informed consent.
10. Has a clinically significant abnormal ECG reading as determined by the investigator, and/or meets the following criteria:
   a. QTc (Friedericia) interval >470 msec. In cases of asymptomatic prolonged QTc interval (>470 msec), the ECG can be repeated up to 2 times. If subsequent QTc interval is <470 msec, the patient may be enrolled but only after review and approval by a cardiologist.
   b. Evidence of Second-Degree AV block type II (Mobitz type II) or AV block type III (complete heart block).
11. Left ventricular ejection fraction (LVEF) less than 50% as measured by echocardiogram at baseline. In cases of LVEF 45-50% in absence of clinical symptoms, after review and clearance by cardiologist, the patient may be enrolled.
12. History of clinically significant cardiac disease including but not limited to the following, within 6 months prior to screening:
   Myocardial infarction
   Unstable angina
   Stroke or transient ischemic attack
   Peripheral arterial disease event
   Heart failure (NYHA class III and IV or ACC/AHA heart failure classification C or D)
13. History of any clinically significant arrhythmia including paroxysmal atrial fibrillation at any time or implantation of a pacemaker or defibrillator.
14. Any history of myocarditis.
15. Signs or symptoms of active angina, arrhythmia or heart failure.
16. Any moderate to severe valve abnormality (stenosis or regurgitation) and/or clinically significant valvular heart disease that has not already been managed surgically.
17. Moderate to large pericardial effusion (e.g., > approximately 100 mL) as measured by echocardiogram at baseline.
18. Patients requiring 2 or more therapeutic paracenteses in the month before screening.
19. Baseline serum troponin above institutional upper limit of normal. In cases of minimally elevated troponin in absence of clinical symptoms, after clearance by a cardiologist, the patient may be enrolled.
20. Ongoing or recent (within 5 years) evidence of significant autoimmune disease that required treatment with systemic immunosuppressive treatments, which may suggest risk for irAEs. The following are not exclusionary: vitiligo, childhood asthma that has resolved, hypothyroidism that required only hormone replacement, type 1 diabetes or psoriasis that does not require systemic treatment.
21. Known history of, or any evidence of interstitial lung disease, or active, non-infectious pneumonitis (past 5 years).
22. Moderate to large pleural effusion as measured by baseline chest X-ray that may require thoracentesis within the next 4 weeks due to size or rate of enlargement. Pre-existing chest tube is acceptable, if patient meets all other inclusion/exclusion criteria.
23. Uncontrolled infection with human immunodeficiency virus, hepatitis B or hepatitis C infection; or diagnosis of immunodeficiency. NOTES:
   Patients with HIV who have controlled infection (undetectable viral load and CD4 count above 350 either spontaneously or on a stable antiviral regimen) are permitted.
   Patients with hepatitis B (HepBsAg+) who have controlled infection (serum hepatitis B virus DNA PCR that is below the limit of detection AND receiving anti-viral therapy for hepatitis B) are permitted.
   Patients who are Hepatitis C virus antibody positive (HCV Ab +) who have controlled infection (undetectable HCV RNA by PCR either spontaneously or in response to a successful prior course of anti-HCV therapy) are permitted.
24. Active infection requiring systemic therapy
25. Receipt of a live vaccine within 30 days of planned start of study medication.
26. Major surgical procedure, open biopsy or significant traumatic injury within 2 weeks prior to enrollment.
27. Prior allogeneic stem cell transplant.
28. Bowel obstruction within the last 3 months or high risk for bowel obstruction (in the opinion of the investigator) or current need for parenteral nutrition.
29. Any medical condition that in the opinion of the investigator would make participation in the study not in the best interest of the patient.
30. Documented allergic or acute hypersensitivity reaction attributed to antibody treatments.
31. Has known allergy or hypersensitivity to components of study drug.
32. Known psychiatric or substance abuse disorders that would interfere with participation with the requirements of the study.
33. Member of the clinical site study team or his/her immediate family.
34. Pregnant or breastfeeding women.
35. Continued sexual activity in women of childbearing potential* who are unwilling to practice highly effective contraception prior to the initial dose/start of the first treatment, during the study, and for at least 6 months after the last dose. Highly effective contraceptive measures include:
   a. stable use of combined (estrogen and progestogen containing) hormonal contraception (oral, intravaginal, transdermal) or progestogen-only hormonal contraception (oral, injectable, implantable) associated with inhibition of ovulation initiated 2 or more menstrual cycles prior to screening;
   b. intrauterine device (IUD); intrauterine hormone-releasing system (IUS);
   c. bilateral tubal ligation;
   d. vasectomized partner (provided that the male vasectomized partner is the sole sexual partner of the WOCBP study participant and that the vasectomized partner has obtained medical assessment of surgical success for the procedure); and/or
   e. sexual abstinence^{†‡}
      * WOCBP are defined as women who are fertile following menarche until becoming postmenopausal, unless permanently sterile. Permanent sterilization methods include hysterectomy, bilateral salpingectomy, and bilateral oophorectomy. A postmenopausal state is defined as no menses for 12 months without an alternative medical cause. A high follicle stimulating hormone (FSH) level in the postmenopausal range may be used to confirm a postmenopausal state in women not using hormonal contraception or hormonal replacement therapy. However, in the absence of 12 months of amenorrhea, a single FSH measurement is insufficient to determine the occurrence of a postmenopausal state. The above definitions are according to Clinical Trial Facilitation Group (CTFG) guidance. Pregnancy testing and contraception are not required for women with documented hysterectomy or tubal ligation.
      † Sexual abstinence is considered a highly effective method only if defined as refraining from heterosexual intercourse during the entire period of risk associated with the study treatments. The reliability of sexual abstinence needs to be evaluated in relation to the duration of the clinical trial and the preferred and usual lifestyle of the patient.
      ‡ Periodic abstinence (calendar, symptothermal, post-ovulation methods), withdrawal (coitus interruptus), spermicides only, and lactational amenorrhoea method (LAM) are not acceptable methods of contraception. Female condom and male condom should not be used together.
   36. Severe and/or uncontrolled hypertension at screening. Patients taking anti-hypertensive medication must be on a stable anti-hypertensive regimen.

### Treatment(s): Monotherapy (Dose, Route, and Schedule)

Dose escalation and dose expansion cohorts: REGN4018 will be administered by once weekly or once every three weeks IV infusion over up to 4 hours ± 15 minutes (including flush).

Cohort exploring SC administration of REGN4018 in week 1 (initial dose) and week 2 (transitional dose): REGN4018 will be administered by once weekly SC injection in weeks 1 and 2. The subsequent doses (including second transitional dose, if applicable, and full doses) will be administered by once weekly IV infusion over up to 4 hours (including flush).

A series of dose escalation cohorts will be used. The escalation scheme beyond DL4a will be chosen based on the tolerability of DL4 and DL4a (see below).

TABLE 5

Dose Escalation Scheme For REGN4018 Monotherapy

| Dose Level | Initial Dose (mg) [Route of Administration] | Transitional Dose (mg) [Route of Administration] | Full Dose (mg) [Route of Administration] | Initial Enrollment |
|---|---|---|---|---|
| DL-1 | 0.03 [IV] | - | 0.1 [IV] | 0 |
| DL1 | 0.1 [IV] | - | 0.3 [IV] | 1 |
| DL2 | 0.3 [IV] | - | 1 [IV] | 1 |
| DL3 | 1 [IV] | - | 3 [IV] | 3-4 |
| DL4 | 1 [IV] | - | 10 [IV] | 3-4 |
| DL4a | 1 [IV] | - | 20 [IV] | 3-4 |
| DL5a | 1 [IV] | 20 [IV] | 60 [IV] | 3-4 |
| DL6a | 1 [IV] | 20 [IV] | 150 [IV] | 3-4 |
| DL7a | 1 [IV] | 20 [IV] | 250 [IV] | 3-4 |
| DL8a | 1 [IV] | 20 [IV] | 450 [IV] | 3-4 |
| DL9a | 1 [IV] | 20 [IV] | 800 [IV] | 3-4 |

### Treatment(s): Combination Therapy (Dose, Route, and Schedule)

Cemiplimab in all combination therapy cohorts: Cemiplimab Q3W 350 mg will be administered by IV infusion over 30 minutes.

REGN4018 in combination therapy cohorts in dose escalation and dose expansion: REGN4018 will be administered by once weekly intravenous (IV) infusion over up to 4 hours ± 15 minutes (including flush). When both drugs are administered on the same day, cemiplimab will be administered first. A series of dose escalation cohorts will be used and will depend on the escalation scheme chosen after additional safety data have been collected.

TABLE 6

Dose Escalation Scheme For REGN4018 and Cemiplimab Combination Therapy

| Dose Level | Initial Dose (mg) [Route of Administration] | Transitional Dose (mg) [Route of Administration] | Full Dose (mg) [Route of Administration] | Cemiplimab Dose [IV] | Initial enrollment |
|---|---|---|---|---|---|
| DLC1 | 0.1 [IV] | - | 0.3 [IV] | 350 mg Q3W | 1 |
| Note: Dose escalation for REGN4018 and cemiplimab combination therapy started at DLC2. | | | | | |
| DLC2 | 0.3 [IV] | - | 1 [IV] | 350 mg Q3W | 1 |
| DLC3 | 1 [IV] | - | 3 [IV] | 350 mg Q3W | 3-4 |
| DLC4 | 1 [IV] | - | 10 [IV] | 350 mg Q3W | 3-4 |
| DLC4a | 1 [IV] | - | 20 [IV] | 350 mg Q3W | 3-4 |
| DLC5a | 1 [IV] | 20 [IV] | 60 [IV] | 350 mg Q3W | 3-4 |
| DLC6a | 1 [IV] | 20 [IV] | 150 [IV] | 350 mg Q3W | 3-4 |
| DLC7a | 1 [IV] | 20 [IV] | 250 [IV] | 350 mg Q3W | 3-4 |
| DLC8a | 1 [IV] | 20 [IV] | 450 [IV] | 350 mg Q3W | 3-4 |
| DLC9a | 1 [IV] | 20 [IV] | 800 [IV] | 350 mg Q3W | 3-4 |

Cohort exploring SC administration of REGN4018 in week 1 (initial dose) and 2 (transitional dose): REGN4018 (2 mg) will be administered by SC injection in week 1 and REGN4018 (25 mg) will be administered subcutaneously in week 2. The subsequent doses (including second transitional dose, if applicable, and full doses) will be administered by once weekly IV infusion over up to 4 hours ± 15 minutes (including flush). Three arm randomized dose expansion will include: REGN4018 (250 mg monotherapy) administered Q3W by IV; REGN4018 (800 mg monotherapy) administered Q3W by IV; and REGN4018 (250 mg or highest tolerable dose in combination with cemiplimab) administered Q3W by IV in combination with cemiplimab (350 mg IV Q3W). In each dose expansion cohort, prior to Q3W administration, patients will receive weekly step-up dosing of REGN4018 over four weeks.

### Study Endpoint(s)

The primary endpoints in the dose escalation phase are dose-limiting toxicities, treatment-emergent adverse events (TEAEs; including immune-related adverse events [irAEs]), serious AEs (SAEs), deaths, laboratory abnormalities (grade 3 or higher per CTCAE), and PK for monotherapy and in combination with cemiplimab.

In the dose expansion phase, the primary endpoint in the study is ORR as measured for both monotherapy and in combination with cemiplimab.

The secondary endpoint in the dose escalation phase is ORR based on Response Evaluation Criteria in Solid Tumors (RESIST 1.1).

The secondary endpoints in the dose expansion phase are:
1) TEAEs; including immune-related, SAEs, deaths, and laboratory abnormalities (grade 3 or higher per CTCAE);
2) Concentration of REGN4018 in serum over time;
3) Change from baseline in QoL as measured by the European Organisation for Research and Treatment of Cancer (EORTC) Quality of Life Questionnaire (QLQ)-C30 GHS/QoL score;
4) Change from baseline in physical functioning as measured by the EORTC QLQ-C30 physical functioning score;
5) Change from baseline in abdominal symptoms as measured by the Measure of Ovarian Symptoms and Treatment (MOST)-Abdominal index score;
6) Time to deterioration in GHS/QoL, physical functioning, and abdominal symptoms; and
7) Change from baseline in QoL as measured by EQ-5D.

The secondary endpoints (in both the dose escalation and dose expansion phases) are:
1) ORR based on iRECIST, best overall response (BOR), duration of response (DOR), disease control rate, CR rate, PFS based on RECIST 1.1 and iRECIST, and CA-125 response; and
2) Presence or absence of anti-drug antibodies against REGN4018 and cemiplimab.

### Procedures and Assessments

The safety and tolerability of REGN4018 alone or in combination with cemiplimab will be monitored by clinical assessment of AEs and by repeated measurements of clinical evaluation including vital signs (temperature, blood pressure, pulse, oxygen saturation, and respiration), physical examinations (complete and limited), 12-lead electrocardiograms (ECGs), echocardiogram, chest x-ray, and laboratory assessment including standard hematology, chemistry and urinalysis.

Because cytokine release following initial dosing (first dose and/or subsequent doses) has been observed with bispecific antibodies and similar molecules, specific measures have been implemented for this study. These measures include: 1 mg initial dose (cycle 1 day 1) at DL4 and above (monotherapy) and DLC3 and above (combination therapy), a transitional dose (cycle 1 day 8) of 20 mg, the option for a split dose at other dosing days, required monitoring on select dose administrations, and use of anti-IL-6 pathway therapy (e.g., tocilizumab) and corticosteroids for management of IRR/CRS. In addition, a safety monitoring scheme will be implemented in the expansion cohorts by including a stopping bound based on the cumulative incidence rate of trigger events (cTE). Enrollment in a given cohort may be paused if the lower bound of the 1-sided 80% confidence interval of the estimated cumulative incidence rate of TE (cTE) excludes 25%. For example, if 4 or more patients out of 11 patients from both dose escalation cohort and expansion cohort experience TE, enrollment will be paused. A discussion between the investigators and the Safety Oversight Committee will decide whether enrollment may be resumed and, if so, whether this will be at the same or lower dose of REGN4018.

A baseline eye examination is required due to expression of MUC16 on the corneal and conjunctival epithelium.

Blood samples for measurement of drug concentration and for ADA assessment will be collected.

Serum and plasma samples will be collected for analysis of additional biomarkers. Exploratory predictive and pharmacodynamic biomarkers related to REGN4018 treatment exposure, clinical activity, or underlying disease will be investigated from collected serum, plasma, whole blood, body fluid, archived tumor tissue, on-study tumor biopsy tissue, tumor DNA (including circulating tumor DNA), and tumor RNA samples.

Anti-tumor activity will be assessed by CT or MRI or PET-CT, and monitoring of performance status and serum CA-125 levels.

Patient-reported outcomes (PROs) will be assessed during the dose expansion phase.

### Statistical Plan

Dose Escalation Phase: There is no formal statistical hypothesis for the dose escalation phase of the study. The analyses of this phase will be descriptive and exploratory in nature. For the dose escalation phase, DLTs observed during the DLT evaluation period will be summarized by dose cohort.

### Dose Expansion Phase:

Statistical hypothesis - For each dose expansion cohort (including each randomized arm in the phase 2 cohort), it is assumed that patients treated with REGN4018 monotherapy or REGN4018/cemiplimab combination therapy will achieve ORR of 25% (H1) or greater. Using 1-sided alpha of 0.05, power of 85% and optimal design, a total of 50 patients with 20 patients for stage 1 are needed for each cohort. Once 20 patients are enrolled in stage 1 for each cohort independently, stage 2 will begin enrollment only if there are 3 or more responders observed over the first 6 months of treatment within the 20 patients enrolled in stage 1. The null hypothesis of 10% will be rejected if 9 or more responses are observed in 50 patients. Confirmed and unconfirmed responses will be combined to determine if the minimum number of responses are achieved to proceed to stage 2.

Primary efficacy analysis - For the given expansion cohort, if the number of responders is greater than or equal to the minimum number of responders specified in the Simon 2-stage design, the treatment is considered as effective and worth further investigation. The ORR will be summarized by descriptive statistics, along with 95% confidence interval. Patients in the safety analysis set (SAF) who are not evaluable for the ORR will be considered as non-responders. The statistical analyses of efficacy for each expansion cohort will be conducted and reported separately, i.e., efficacy results and clinical conclusions from each cohort will not affect the other cohorts, and vice versa.

Safety observations and measurements including drug exposure, AEs, laboratory data, and vital signs will be summarized.

Preliminary Results: Initial safety, pharmacokinetics (PK), and efficacy data from the REGN4018 (ubamatamab) monotherapy portion of the first-in-human Phase 1 dose-escalation study in patients with recurrent ovarian cancer is presented below.

Patients with recurrent platinum-experienced and/or -intolerant ovarian cancer with elevated cancer antigen (CA)-125 levels were administered REGN4018 intravenously (IV) weekly, at a dose range of 0.1-800 mg. Dose escalation followed a modified 3+3 (4+3) design. The dose-limiting toxicity (DLT) period was 28-35 days. Step-up dosing for the initial two doses was utilized to mitigate risk of cytokine release syndrome (CRS) via gradual increase of drug exposure. Primary endpoints included safety and PK. Secondary endpoints included preliminary efficacy as determined by objective response rate (ORR) per Response Evaluation Criteria in Solid Tumors (RECIST) 1.1.

Seventy eight patients received REGN4018 monotherapy in the phase 1 portion of the study; median duration of exposure was 12 (range, 0.4-117) weeks. Based on histology, the 78 patients included: 71 (91.0%) high-grade serous, 2 (2.6%) clear cell, 1 (1.3%) high-grade endometroid, 1 (1.3%) low-grade serous, and 3 (3.8%) other. CA-125 baseline serum levels ranged from 107-10000, with a median of 709 U/mL. Of the 78 patients, 26 (33%) had visceral metastases, and 30 (58%) had >75% tumor cells with 2+ baseline MUC16 IHC staining. The median number of prior therapies was 4.5 (range, 1-17). The most common treatment-emergent adverse events (TEAE) were CRS (73.1%, all Grade 1-2) and pain (87.2%, most Grade 1-2); these primarily occurred during weeks 1-2 of initial step-up dosing. The most common Grade ≥3 TEAEs were anaemia (23.1%) and abdominal pain (19.2%). Objective responses were observed between 20-800 mg doses (n=50). ORR of those receiving ≥1 full dose (n=42) was 14.3% (95% CI 5.4-28.5) and disease control rate (DCR) was 57.1% (41.0-72.3). In a subset of these without baseline visceral metastases (n=29) ORR was 20.7% (8.0-39.7) and DCR was 72.4% (52.8-87.3). Median duration of response was 12.2 months. Exploratory analysis of patients with >75% of tumour cells with 2+ baseline MUC16 immunohistochemical staining and ≥1 dose of ≥20 mg (n=13) showed ORR of 30.8% (9.1-61.4), and DCR 61.5% (31.6-86.1). 46.2% of patients with >75% of tumor cells with 2+ baseline MUC16 IHC staining showed a CA-125 response. Serum ubamatamab concentrations increased dose-proportionally, and showed linear pharmacokinetics. No definitive doseresponse relationship was observed in safety or efficacy between 20-800 mg.

REGN4018 safety profile was acceptable with evidence of durable responses in this heavily pretreated population with ovarian cancer across a wide dose range. Data from this analysis supports further investigation of REGN4018 in recurrent platinum-experienced ovarian cancer.

In phase 2, up to 150 patients with advanced platinum-resistant OC and elevated serum CA-125 will be randomised to three IV arms (1:1:1) to receive ubamatamab (REGN4018) 250 mg IV Q3W or 800 mg IV Q3W as monotherapy, or ubamatamab 250 mg IV Q3W in combination with cemiplimab 350 mg Q3W. All arms will include weekly step-up dosing of ubamatamab (1 mg week 1, 20 mg week 2, and full dose weeks 3 and 4) to limit risk of cytokine release syndrome prior to proceeding to Q3W dosing. Expansion cohorts will use a Simon 2-stage study design, with an interim analysis after the first 20 patients. Any arm with ≥3 partial responses or better will be expanded to 50 patients.

In this dose expansion phase the primary endpoint will be the objective response rate for each arm as defined by RECIST 1.1 criteria. Secondary endpoints include evaluation of duration of response and progression-free survival as well as further evaluation of safety and pharmacokinetics. Exploratory endpoints include evaluation of baseline tumour MUC16 immunohistochemistry expression and other biomarkers as predictors of response. The impact of ubamatamab on quality of life and physical functioning will also be assessed.

In some cases, the 250 mg dose may be split into two fractions, the first fraction including 50 mg and the second fraction including 200 mg.

### Example 10: Translational Findings From the Study of REGN4018 in Patients With Recurrent Ovarian Cancer

Intravenous ubamatamab was administered to patients with ovarian cancer as monotherapy (78 pts) or in combination with anti-PD-1 cemiplimab (27 pts) in the phase 1 study detailed in Example 9. The first step-up dose in Week (W) 1 was selected based on in vitro cytokine assay results. Mouse tumor regression models suggested effective concentrations to suppress tumor growth. Pharmacokinetic (PK) data in monkeys were scaled to predict drug exposures. Modeling of CD3 bispecific antibody + cemiplimab was used to set dosing sequence of cemiplimab plus ubamatamab. Preclinical and clinical PK, cytokine, and efficacy data from dose escalation were integrated to determine regimens in dose expansion. Population PK modeling simulated regimens of interest.

In vitro data showed min and max cytokine release at concentrations of 0.01 mg/L and ≥0.1 mg/L. Data supported W1 step-up doses in the range of 0.1-1 mg, leading to observed Cmax of 0.02-0.3 mg/L. Cmax values at W1 in all cohorts in patients were well predicted (within ±30%) from the scaling. PK data from mouse tumor regression model indicated efficacious concentrations (0.5-50 mg/L) that aligned with Cmin >4 mg/L at effective doses of ≥20 mg. PK simulation supported testing ≥250 mg Q3W in expansion. As the model predicted, no significant cytokine release occurred when combining cemiplimab with ubamatamab following an ubamatamab monotherapy lead-in.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

TABLE 7

Sequences Excluded from ST.26-Formatted Sequence Listing

| SEQ ID NO: | Sequence |
|---|---|
| 12 | TAS |
| 39 | AAS |

What is claimed is:

1. A method of treating a MUC16-expressing cancer in a subject in need thereof, comprising administering to the subject a bispecific antibody comprising a first antigen-binding domain that specifically binds mucin 16 (MUC16) on a target tumor cell, and a second antigen-binding domain that specifically binds human CD3 on a T cell, wherein the bispecific antibody is administered to the subject at a dose of at least 1 mg;
wherein the first antigen-binding domain comprises a heavy chain variable region (HCVR) comprising three heavy chain complementarity determining regions (HCDR1, HCDR2 and HCDR3), and a light chain variable region (LCVR) comprising three light chain complementarity determining regions (LCDR1, LCDR2 and LCDR3), wherein the HCDR1, HCDR2, HCDR3, LCDR1, LCDR2, and LCDR3 comprise, respectively, the amino acid sequences of SEQ ID NOs: 8, 9, 10, 11, 12, and 13; and
wherein the second antigen-binding domain comprises a heavy chain variable region (HCVR) comprising three heavy chain complementarity determining regions (HCDR1, HCDR2 and HCDR3), and a light chain variable region (LCVR) comprising three light chain complementarity determining regions (LCDR1, LCDR2 and LCDR3), wherein the HCDR1, HCDR2, HCDR3, LCDR1, LCDR2, and LCDR3 comprise, respectively, the amino acid sequences of SEQ ID NOs: 14, 15, 16, 11, 12, and 13.

2. The method of claim 1, wherein the cancer is ovarian cancer, fallopian tube cancer, or primary peritoneal cancer.

3. The method of claim 1, wherein the cancer is resistant to platinum-based chemotherapy.

4. The method of claim 1, wherein the subject has previously been treated with a platinum-based chemotherapy.

5-7. (canceled)

8. The method of claim 1, wherein the first antigen-binding domain comprises a HCVR comprising the amino acid sequence of SEQ ID NO: 1, and a LCVR comprising the amino acid sequence of SEQ ID NO: 2.

9-11. (canceled)

12. The method of claim 8, wherein the second antigen-binding domain comprises a HCVR comprising the amino acid sequence of SEQ ID NO: 3, and a LCVR comprising the amino acid sequence of SEQ ID NO: 2.

13. The method of claim 12, wherein the bispecific antibody comprises a human IgG heavy chain constant region.

14. The method of claim 13, wherein the human IgG heavy chain constant region is isotype IgG1.

15. The method of claim 13, wherein the human IgG heavy chain constant region is isotype IgG4.

16. The method of claim 13, wherein the bispecific antibody comprises a chimeric hinge that reduces Fcy receptor binding relative to a wild-type hinge of the same isotype.

17. The method of claim 13, wherein the first heavy chain or the second heavy chain, but not both, comprises a CH3 domain comprising a H435R (EU numbering) modification and a Y436F (EU numbering) modification.

18. The method of claim 1, wherein the bispecific antibody comprises a first heavy chain comprising the amino acid sequence of SEQ ID NO: 29.

19. The method of claim 1, wherein the bispecific antibody comprises a second heavy chain comprising the amino acid sequence of SEQ ID NO: 31.

20. The method of claim 1, wherein the bispecific antibody comprises a first heavy chain comprising the amino acid sequence of SEQ ID NO: 29, a second heavy chain comprising the amino acid sequence of SEQ ID NO: 31, and a common light chain comprising the amino acid sequence of SEQ ID NO: 30.

21. The method of claim 1, wherein the subject has a serum CA-125 level equal to or greater than 60 U/ml.

22. The method of claim 1, further comprising administering a second therapeutic agent or therapeutic regimen.

23. The method of claim 22, wherein the second therapeutic agent or therapeutic regimen comprises an anti-PD-1 antibody or antigen-binding fragment thereof.

24. The method of claim 23, wherein the anti-PD-1 antibody or antigen-binding fragment comprises:
(a) three heavy chain complementarity determining regions (HCDR1, HCDR2 and HCDR3) contained within a heavy chain variable region (HCVR) comprising the amino acid sequence of SEQ ID NO: 33; and
(b) three light chain complementarity determining regions (LCDR1, LCDR2 and LCDR3) contained within a light chain variable region (LCVR) comprising the amino acid sequence of SEQ ID NO: 34.

25. The method of claim 24, wherein the anti-PD-1 antibody or antigen-binding fragment comprises a HCDR1 comprising the amino acid sequence of SEQ ID NO: 35, a HCDR2 comprising the amino acid sequence of SEQ ID NO: 36, and a HCDR3 comprising the amino acid sequence of SEQ ID NO: 37.

26. The method of claim 24, wherein the anti-PD-1 antibody or antigen-binding fragment comprises a LCDR1 comprising the amino acid sequence of SEQ ID NO: 38, a LCDR2 comprising the amino acid sequence of SEQ ID NO: 39, and a LCDR3 comprising the amino acid sequence of SEQ ID NO: 40.

27. The method of claim 24, wherein the anti-PD-1 antibody or antigen-binding fragment comprises a HCVR comprising the amino acid sequence of SEQ ID NO: 33, and a LCVR comprising the amino acid sequence of SEQ ID NO: 34.

28. The method of claim 27, wherein the anti-PD-1 antibody or antigen-binding fragment is an anti-PD-1 antibody comprising a heavy chain comprising the amino acid sequence of SEQ ID NO: 41 and a light chain comprising the amino acid sequence of SEQ ID NO: 42.

29. The method of claim 1, wherein the bispecific antibody is administered in a dosing regimen comprising a split initial dose.

30. The method of claim 1, wherein the bispecific antibody is administered to the subject at a dose of from 10 mg to 1000 mg weekly.

31. The method of claim 30, wherein the bispecific antibody is administered to the subject at a dose of about 250 mg weekly, optionally wherein the dose is split into a first fraction of about 50 mg, and a second fraction of about 200 mg.

32. The method of claim 30, wherein the bispecific antibody is administered to the subject at a dose of about 800 mg weekly, optionally wherein the dose is split into a first fraction of about 50 mg, and a second fraction of about 750 mg.

33. The method of claim 1, wherein the bispecific antibody is administered to the subject at a dose of from 10 mg to 1000 mg once every three weeks.

34. The method of claim 33, wherein the bispecific antibody is administered to the subject at a dose of about 250 mg once every three weeks, optionally wherein the dose is split into a first fraction of about 50 mg, and a second fraction of about 200 mg.

35. The method of claim 33, wherein the bispecific antibody is administered to the subject at a dose of about 800 mg once every three weeks, optionally wherein the dose is split into a first fraction of about 50 mg, and a second fraction of about 750 mg.

36. The method of claim 30, wherein the bispecific antibody is administered in a dosing regimen comprising: (i) administering 1 mg of the bispecific antibody in week 1, optionally wherein the dose is split into a first fraction of about 0.5 mg and a second fraction of about 0.5 mg; (ii) administering 20 mg of the bispecific antibody in week 2, optionally wherein the dose is split into a first fraction of about 10 mg and a second fraction of about 10 mg; and (iii) administering 250 mg of the bispecific antibody in week 3, optionally wherein the dose is split into a first fraction of about 50 mg, and a second fraction of about 200 mg.

37. The method of claim 36, further comprising administering the bispecific antibody at a dose of about 250 mg once every week from week 4 onwards.

38. The method of claim 36, further comprising administering the bispecific antibody at a dose of about 250 mg once every three weeks from week 4 onwards.

39. The method of claim 36, further comprising administering the bispecific antibody at a dose of about 800 mg once every three weeks from week 4 onwards.

40. The method of claim 23, wherein the anti-PD-1 antibody is administered to the subject at a dose of from 300 to 400 mg once every three weeks.

41. The method of claim 40, wherein the anti-PD-1 antibody is administered to the subject at a dose of 350 mg once every three weeks.

42. The method of claim 1, wherein the subject has stable disease, a partial response, or a complete response following administration of the bispecific antibody for at least one week at a dose of 1-800 mg.

43. The method of claim 1, wherein the subject has stable disease, a partial response, or a complete response following administration of the bispecific antibody for at least one week at a dose of 20-800 mg.

44. The method of claim 1, wherein the bispecific antibody is administered to the subject at a dose sufficient to achieve a serum concentration of at least 4 mg/L.

45. The method of claim 1, wherein MUC16 is highly expressed in ≥75% of tumor cells in the subject, as determined by immunohistochemical staining.

46. The method of claim 1, wherein the subject has:
▪ a baseline MUC16 immunohistochemical staining score of 2 in a MUC16-expressing tumor; or
▪ a baseline MUC16 immunohistochemical staining score of 2+ in a MUC16-expressing tumor; or
▪ a baseline MUC16 immunohistochemical staining score of 3 in a MUC16-expressing tumor; or
▪ a baseline MUC16 immunohistochemical staining score of 3+ in a MUC16-expressing tumor; or
▪ a baseline MUC16 immunohistochemical staining score of 4 in a MUC16-expressing tumor; or
▪ a baseline MUC16 immunohistochemical staining score of 4+ in a MUC16-expressing tumor; or
▪ a baseline MUC16 immunohistochemical staining score of 5 in a MUC16-expressing tumor; or
▪ a tumor with MUC16-expression in ≥ 50% of tumor cells; or
▪ a tumor with MUC16-expression in ≥ 55% of tumor cells; or
▪ a tumor with MUC16-expression in ≥ 60% of tumor cells; or
▪ a tumor with MUC16-expression in ≥ 65% of tumor cells; or
▪ a tumor with MUC16-expression in ≥ 70% of tumor cells; or
▪ a tumor with MUC16-expression in ≥ 75% of tumor cells.

47. The method of claim 1, wherein the bispecific antibody is administered intravenously.

48. The method of claim 1, wherein the bispecific antibody is administered subcutaneously.

49. The method of claim 23, wherein the anti-PD-1 antibody or antigen-binding fragment is administered intravenously.


SEQUENCE LISTING (42 sequences)

SEQ ID NO: 1 - protein, 117 aa, synthetic construct
  REGION 1..117: note = anti-MUC16 HCVR
QVQLVESGGGLVKPGGSLRLSCAASGFTFSNYYMSWVRQAPGKGLEWISYISGRGSTIFYADSVKGRITISRDNAKNSLFLQMNSLRAEDTAVYFCVKDRGGYSPYWGQGTLVTVSS

SEQ ID NO: 2 - protein, 108 aa, synthetic construct
  REGION 1..108: note = anti-MUC16 and anti-CD3 LCVR
DIQMTQSPSSLSASVGDRVTITCRASQSISTYLNWYQQKPGKAPKLLIYTASSLQSGVPSRFSGSGSGTDFTLTISSLQPEDFATYYCQQSYSTPPITFGQGTRLEIK

SEQ ID NO: 3 - protein, 124 aa, synthetic construct
  REGION 1..124: note = CD3-VH-G
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSS

SEQ ID NO: 4 - protein, 124 aa, synthetic construct
  REGION 1..124: note = CD3-VH-G5
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKDGSGYGKFYYYGMDVWGQGTTVTVSS

SEQ ID NO: 5 - protein, 124 aa, synthetic construct
  REGION 1..124: note = CD3-VH-G9
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKDGSGYGKFYHYGLDVWGQGTTVTVSS

SEQ ID NO: 6 - protein, 124 aa, synthetic construct
  REGION 1..124: note = CD3-VH-G10
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYYYGLDVWGQGTTVTVSS

SEQ ID NO: 7 - protein, 124 aa, synthetic construct
  REGION 1..124: note = CD3-VH-G20
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKYGSGYGKFYYYGMDVWGQGTTVTVSS

SEQ ID NO: 8 - protein, 8 aa, synthetic construct
  REGION 1..8: note = anti-MUC16 HCDR1
GFTFSNYY

SEQ ID NO: 9 - protein, 8 aa, synthetic construct
  REGION 1..8: note = anti-MUC16 HCDR2
ISGRGSTI

SEQ ID NO: 10 - protein, 10 aa, synthetic construct
  REGION 1..10: note = anti-MUC16 HCDR3
VKDRGGYSPY

SEQ ID NO: 11 - protein, 6 aa, synthetic construct
  REGION 1..6: note = anti-MUC16 and anti-CD3 LCDR1
QSISTY

SEQ ID NO: 12 - skipped

SEQ ID NO: 13 - protein, 10 aa, synthetic construct
  REGION 1..10: note = anti-MUC16 and anti-CD3 LCDR3
QQSYSTPPIT

SEQ ID NO: 14 - protein, 8 aa, synthetic construct
  REGION 1..8: note = CD3-VH-G HCDR1
GFTFDDYS

SEQ ID NO: 15 - protein, 8 aa, synthetic construct
  REGION 1..8: note = CD3-VH-G HCDR2
ISWNSGSK

SEQ ID NO: 16 - protein, 17 aa, synthetic construct
  REGION 1..17: note = CD3-VH-G HCDR3
AKYGSGYGKFYHYGLDV

SEQ ID NO: 17 - protein, 8 aa, synthetic construct
  REGION 1..8: note = CD3-VH-G5 HCDR1
GFTFDDYS

SEQ ID NO: 18 - protein, 8 aa, synthetic construct
  REGION 1..8: note = CD3-VH-G5 HCDR2
ISWNSGSI

SEQ ID NO: 19 - protein, 17 aa, synthetic construct
  REGION 1..17: note = CD3-VH-G5 HCDR3
AKDGSGYGKFYYYGMDV

SEQ ID NO: 20 - protein, 8 aa, synthetic construct
  REGION 1..8: note = CD3-VH-G9 HCDR1
GFTFDDYS

SEQ ID NO: 21 - protein, 8 aa, synthetic construct
  REGION 1..8: note = CD3-VH-G9 HCDR2
ISWNSGSK

SEQ ID NO: 22 - protein, 17 aa, synthetic construct
  REGION 1..17: note = CD3-VH-G9 HCDR3
AKDGSGYGKFYHYGLDV

SEQ ID NO: 23 - protein, 8 aa, synthetic construct
  REGION 1..8: note = CD3-VH-G10 HCDR1
GFTFDDYS

SEQ ID NO: 24 - protein, 8 aa, synthetic construct
  REGION 1..8: note = CD3-VH-G10 HCDR2
ISWNSGSK

SEQ ID NO: 25 - protein, 17 aa, synthetic construct
  REGION 1..17: note = CD3-VH-G10 HCDR3
AKYGSGYGKFYYYGLDV

SEQ ID NO: 26 - protein, 8 aa, synthetic construct
  REGION 1..8: note = CD3-VH-G20 HCDR1
GFTFDDYS

SEQ ID NO: 27 - protein, 8 aa, synthetic construct
  REGION 1..8: note = CD3-VH-G20 HCDR2
ISWNSGSI

SEQ ID NO: 28 - protein, 17 aa, synthetic construct
  REGION 1..17: note = CD3-VH-G20 HCDR3
AKYGSGYGKFYYYGMDV

SEQ ID NO: 29 - protein, 443 aa, synthetic construct
  REGION 1..443: note = anti-MUC16 HCaa 1-117: Variable regionaa 118-443: Constant region
QVQLVESGGGLVKPGGSLRLSCAASGFTFSNYYMSWVRQAPGKGLEWISYISGRGSTIFYADSVKGRITISRDNAKNSLFLQMNSLRAEDTAVYFCVKDRGGYSPYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 215 aa, synthetic construct
  REGION 1..215: note = anti-MUC16 and anti-CD3 LCaa 1-108: Variable regionaa 109-215: Constant region
DIQMTQSPSSLSASVGDRVTITCRASQSISTYLNWYQQKPGKAPKLLIYTASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 450 aa, synthetic construct
  REGION 1..450: note = anti-CD3-G HCaa 1-124: Variable regionaa 125-450: Constant region
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 32 - protein, 450 aa, synthetic construct
  REGION 1..450: note = anti-CD3-G20 HCaa 1-124: Variable regionaa 125-450: Constant region
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKYGSGYGKFYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 33 - protein, 117 aa, synthetic construct [sequence omitted]

SEQ ID NO: 34 - protein, 107 aa, synthetic construct [sequence omitted]

SEQ ID NO: 35 - protein, 8 aa, synthetic construct
  REGION 1..8: note = anti-PD-1 HCDR1
GFTFSNFG

SEQ ID NO: 36 - protein, 8 aa, synthetic construct
  REGION 1..8: note = anti-PD-1 HCDR2
ISGGGRDT

SEQ ID NO: 37 - protein, 10 aa, synthetic construct
  REGION 1..10: note = anti-PD-1 HCDR3
VKWGNIYFDY

SEQ ID NO: 38 - protein, 6 aa, synthetic construct
  REGION 1..6: note = anti-PD-1 LCDR1
LSINTF

SEQ ID NO: 39 - skipped

SEQ ID NO: 40 - protein, 9 aa, synthetic construct
  REGION 1..9: note = anti-PD-1 LCDR3
QQSSNTPFT

SEQ ID NO: 41 - protein, 444 aa, synthetic construct
  REGION 1..444: note = anti-PD-1 HCaa 1-117: Variable regionaa 118-444: Constant region
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 214 aa, synthetic construct
  REGION 1..214: note = anti-PD-1 LCaa 1-108: Variable regionaa 109-214: Constant region
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC